SEQUENCE LISTING (2 sequences)

SEQ ID NO: 1 - protein, 133 aa, Homo sapiens
APTSSSTRKTQLQLEHLLLDLQMILNGINNYKNPKLTRMLTFKFYMPWKATELKHLQCLEEELKPLEEVLNLAQSKNFHLRPRDLISNINVIVLELKGSETTFMCEYADETATIVEFLNRWITFAQSIISTLT

SEQ ID NO: 2 - DNA, 405 bp, synthetic construct
  source 1..405: mol_type = other DNA
gcacctacttcaagttctacacggaaaacacagctacaactggagcatttactgctggatttacagatgattttgaatggaattaataattacaagaatcccaaactcaccaggatgctcacatttaagttttacatgccctggaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattacctttgctcaaagcatcatctcaacactgacttaataa

(19) United States
(12) Patent Application Publication
(10) Pub. No.: US 2025/0154217 A1
(43) Pub. Date: May 15, 2025

(54) METHOD FOR REALIZING CONTROLLED RELEASE AND SUSTAINED RELEASE OF ACTIVITY OF BIOACTIVE MOLECULES AND USE FOR DRUGS

(71) Applicant: ZONHON BIOPHARMA INSTITUTE, INC., Changzhou, Jiangsu (CN)

(72) Inventors: Bruce Yong MA, Changzhou, Jiangsu (CN); Jianhua HANG, Changzhou, Jiangsu (CN); Baixue ZHAO, Changzhou, Jiangsu (CN); Zhen XU, Changzhou, Jiangsu (CN); He WANG, Changzhou, Jiangsu (CN); Jun WANG, Changzhou, Jiangsu (CN); Peipei CAO, Changzhou, Jiangsu (CN); Chenyang JIANG, Changzhou, Jiangsu (CN); Yu ZHUANG, Changzhou, Jiangsu (CN)

(21) Appl. No.: 18/839,590

(22) PCT Filed: Feb. 17, 2023
(86) PCT No.: PCT/CN2023/076746

§ 371 Date: Aug. 19, 2024

(30) Foreign Application Priority Data
Feb. 18, 2022 (CN) 202210153497.5

(51) Int. Cl. C07K 14/55; A61K 45/06; A61K 47/54; A61K 47/60; A61P 35/00

(52) U.S. Cl. CPC C07K 14/55; A61K 45/06; A61K 47/545; A61K 47/60; A61P 35/00

(57) ABSTRACT

Provided is a method for realizing the controlled release and sustained release of the activity of bioactive molecules and use for drugs. Provided is a PEG modifier of a specific structure. The PEG modifier can be widely applied to the controlled release and sustained release of the activity of various bioactive molecules. A conjugate is formed by reacting the PEG modifier of the specific type and structure with IL-2. In a certain in vitro condition or in vivo physiological condition, PEG gradually falls off from the conjugate and the activity of IL-2 is gradually released, so that a certain effective plasma concentration where bioactive molecules are relatively stable can be maintained, and thus the in vivo or in vitro sustained release and controlled release of the activity of the drug are realized.

### TECHNICAL FIELD

The invention relates to a method for achieving controlled release and sustained release of bioactive molecules and use for drugs, in particular to highly active cytokines, such as the method for achieving controlled release and sustained release of interleukin-2.

### BACKGROUND OF THE INVENTION

Interleukin-2 (IL-2) is a member of the large family of interleukins. A large number of studies have shown that: IL-2 can promote the differentiation and maturation of T cells, NK cells and B cells, activate their biological activities; induce the activation of killer cells (LAK) activated by lymphokines; and promote the synthesis and release of many lymphokines such as interferon and tumor necrosis factor, as well as the generation of antibodies. The function of expansion of lymphoid cells in the body and effector function improvement of these cells confers antitumor effect of IL-2. In the early 1980 s, high dose of IL-2 has been approved for the treatment of metastatic renal cell carcinoma and malignant melanoma.

However, IL-2 has a short half-life in vivo and low drug availability, which often requires frequent and repeated administration to maintain efficacy in clinical applications. On the other hand, human IL-2 inevitably activates a large number of Treg cells by binding to the human IL-2 high-affinity receptor IL-2Rαβγ(Kd:10⁻¹¹). These two aspects result in varying degrees of side effects together. The most serious is Vascular Leakage Syndrome (VLS), which leads to the accumulation of intravascular fluid in organs such as liver and lung, and then causes pulmonary edema and liver cell damage, so that patients have to withdraw the treatment, which greatly reduces the compliance of patients and limits the further clinical application of related therapies.

Researchers often use water-soluble polymers such as polyethylene glycol to modify drugs, to extend the biological half-life of drugs, reduce the immunogenicity and toxicity of drugs (Nandini V, Proc. Natl. Acad. Sci, 1987; Chang Yuan, PEGylation of interleukin-2, 1996). Wang Lifu et al. modified wild-type rIL-2 with a monomethoxy-polyethylene glycol active ester with purity of 75% and molecular weight of 5000, and the randomly modified product retained only 69.7% in vitro activity (Wang Lifu et al. Preparation of polyethylene glycol modified rIL-2 and its anti-hepatoma cell effect in vitro and in vivo, 1997). In addition, it is not ideal to realize the slow release of drugs in vivo and improve the bioavailability of drugs through the conventional polyethylene glycol modification method. In the prior art, it is also reported that water-soluble polymers and drugs are linked by a linker to form polymer-drug conjugates. The release of water-soluble polymer from the conjugate can achieve the purpose of sustained and controlled release of drug activity. The drug stays in the lesion (such as cancer) for a longer time, which can reduce the frequency of drug administration and reduce the inconvenience of patient. Patent CN200680029849.5 exposes a yoke compound, contains aromatic moiety containing ionizable hydrogen atoms such as fluorene, interval and water-soluble polymer. Patent CN103517718A further exposes the conjugate formed by the polymer with rIL-2. This conjugate is actually a CD122 (IL-2Rβ)-biased agonist NKTR-214 launched by Nektar Corporation of the United States. The conjugate is obtained by conjugating six branched PEG with a molecular weight of 20K and with fluorene ring structure to IL-2 (with the same amino acid sequence as aldesleukin). The conjugate removes the fluorenylmethoxycarbonyl group by β-elimination reaction under mild alkaline conditions. Thus, NKTR-214 can gradually release the five PEGs on the surface of the protein under physiological conditions (pH=7.4, weak alkaline), giving NKTR-214 the ability to preferentially bind to IL-2Rβ receptor to enhance the activation of T cells and circulate in vivo for a long time. In the process of preparation of NKTR-214, the modification of lysine residues (such as K31, K34, K42, K47, K48, K75) that accumulated at the IL-2/IL-2Rα interface was promoted by optimizing PEG reagents and coupling reaction. PEG was located near the key hydrophobic binding site of IL-2/IL-2Rα interaction to reduce the binding of CD25 (IL-2Rα) and preferentially activate CD122 (IL-2Rβ) (Charych D H, Hoch U, Langowski J L, et al. NKTR-214, an engineered cytokine with biased IL2 receptor binding, increased tumor exposure, and marked efficacy in mouse tumor models[J]. Clinical Cancer Research An Official Journal of the American Association for Cancer Research, 2016, 22 (3): 680-690.). Nektar and BMS had a $3.6 billion partnership for NKTR214. However, two phase III clinical studies, PIVOT IO 001 and PIVOT-09, failed in 2022, with treatment data not meeting the primary endpoint (2022 EMSO, Annual Meeting of European Society of Medical Oncology, Abstract No. 7850, LBA68).

After extensive testing and research, the inventors of the invention have selected a specific structural PEG modifier among existing mature polyethylene glycol modifiers, which can be used to modify IL-2 or its variants, so that it can obtain excellent controlled release and sustained release effect of activity, achieve prominent advantages in the bioavailability compared with related drugs, reduce the frequency of administration, and greatly improve the therapeutic effect of drugs and patient compliance. At the same time, the present invention is also different from NKTR-214 in molecular design. After administration, the PEG molecule in NKTR-214 drug has a very complex and uncontrollable dissociation behavior in vivo, so it cannot always maintain the characteristics of CD122 biased activation theoretically. Thus, it will affect the stability of the drug effect. Through site-directed mutagenesis of the proprotein sequence, the inventor achieved the bias of CD122 and the controllability of PEG modification sites, which solved the potential shortcomings of NKTR-214 in molecular design from three aspects of safety, efficacy and quality controllability. This differential advantage was clarified by pharmacokinetics and pharmacodynamics data in vivo.

### SUMMARY OF THE INVENTION

In order to overcome the problem of low drug utilization rate of biological active molecules such as IL-2 and the generally reduced activity of polyethylene glycol modified product, the invention provides a method for realizing the controlled release and sustained release of the activity of biological active molecules. Polyethylene glycol modifier with specific structure is reacted with biological active molecules such as IL-2 to form a conjugate. Under certain in vitro or in vivo physiological conditions, PEG fall off from the conjugate and the activity of bioactive molecules is gradually released. Thus, the relatively stable and effective blood drug concentration of the active molecules is maintained, controlled and sustained release of the drug in vivo and in vitro is achieved, better dynamic balance of drug bioavailability is achieved, and has clinical application prospects such as reducing the frequency of administration, improving the bioavailability of the drug, better patient compliance and safety. On the other hand, this application provides a new kind of IL-2 mutant proteins, which is mutated at the PEG modification site, making the PEG modification more controllable. Moreover, this mutant is a mutation in the binding site of IL-2 to its receptor, and its activity is higher than that of wild type IL-2.

An object of the present invention is to provide a method for achieving controlled and sustained release of the activity of bioactive molecule, the method mentioned above is to modify the amino group of bioactive molecules with polyethylene glycol modifier to obtain modified product; as the PEG gradually fals off from the modified product, the activity of bioactive molecules is gradually released, and the polyethylene glycol modifier is polyethylene glycol succinimidyl succinate.

Described bioactive molecules are proteins or peptides, optimization of interleukin, the most optimization of IL-2.

The PEG modifier is preferably straight-chain polyethylene glycol succinimidyl succinate.

A further object of the present invention is to provide polyethylene glycol modified human IL-2, the PEG modifier used is polyethylene glycol succinimidyl succinate, 4.5 to 8.5 PEG molecules are conjugated to one IL2 molecule on average.

Preferably, the polyethylene glycol modifier is straight-chain polyethylene glycol succinimidyl succinate.

Further preferably, molecular weight of the PEG modifier is 5 to 20 kDa. Described molecular weight is marked value, the actual molecular weight can be 90% to 1¹⁰% of marked value. When the molecular weight of the PEG modifier was 5 kDa, 5.5 to 7.5 PEG molecules are conjugated to one human interleukin-2 molecular on average. When the molecular weight of PEG modifier is 10 to 20 kDa, 6.5 to 8.5 PEG molecules are conjugated to one human interleukin-2 molecular on average.

Further preferably, the structure of PEG modifier is shown as follows:

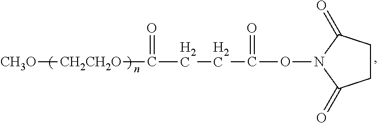

wherein n is an integer from 97 to 494. When n is an integer from 97 to 494, the actual molecular weight of the PEG modifier is about 4.5 k-22 k, and labeling value of the corresponding molecular weight is 5-20 kDa.

Further preferably, the structure of polyethylene glycol modified human IL-2 is as follows:

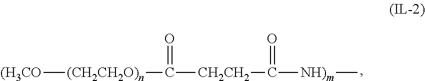

wherein n is an integer from 97 to 494, and m is 4.5 to 8.5.

A further aim of the present invention is to provide a human IL-2 mutant, in the first aspect, it does not contain adjacent lysine and the PEG modified product is more homogeneous. Inventor found amino acid at position 8, 9, 48, 49 from the N-terminus of the wild type IL-2 shown as SEQ ID NO: 1 are lysine, which could be PEG modified, and the adjacent site will hardly be PEG modified at the same time. When IL-2 are saturated modified, position 8 or 9 and position 48 or 49 may be modified, therefor four different modification site isomers might be produced, resulting in the heterogeneous modified human IL-2. Accordingly, the inventor chose to mutate amino acids at position 8 and/or 9, 48 and/or 49 to greatly reduce the generation of PEG modification site isomers and facilitate the homogeneity control of PEG modified product. Before this application, skilled in the art were not aware of the possible adverse effects on PEG modification when the adjacent amino acids on the IL-2 sequence were lysine, so there was no motivation to mutate the corresponding sites when IL-2 was modified with polyethylene glycol.

The present invention provides human IL-2 mutants, amino acid at position 8 or/and 9 from the N-terminus of wild-type human IL-2 is substituted; or amino acid at position 48 or/and 49 from the N-terminus of wild-type human IL-2 is substituted; the amino acid sequence of the wild-type human IL-2 is shown as SEQ ID NO: 1.

Preferably, amino acids at position 8 or/and 9, and position 48 or/and 49 from the N-terminus of wild-type human IL-2 is substituted.

Specific embodiments include mutation of lysine at position 8 to arginine, mutation of lysine at position 9 to arginine, mutation of lysine at position 48 to arginine, tryptophan or tyrosine, and mutation of lysine at position 49 to aspartic acid. But the embodiments are intended only as examples, rather than limit the scope of the present invention. The purpose of mutations at position 8, 9, 48 and 49 is to solve the problem that wild type IL-2 has adjacent lysines, which results in the production of multiple PEG modified isomers and affected the homogeneity of PEG modified products. Accordingly, one skilled in the art knows that mutation to other non-lysine amino acids other than the amino acids mentioned in examples also can achieve the above purpose.

In the second aspect, the human IL-2 mutant without adjacent lysine was further optimized to obtain a mutant with significantly better in vitro activity than wild type IL-2. The mutation is that one, two or more amino acids at position 1, 8, 9, 18, 19, 48, 49, 72 or/and 81 from the N-terminus of wild-type human IL-2 are substituted; the amino acid sequence of the wild-type human IL-2 is shown as SEQ ID NO: 1.

Further preferably, the mutation comprises one or more substitutions selected from the group consisting of: position 1: A1 deletion; position 8: K8R; position 9: K9R; position 18: L18M; position 19: L19S; position 48: K48W, R; position 49: K49R; position 72: L72F; position 81: R81D. More preferably, the mutant comprises one or more mutations selected from the following: position 8: K8R; position 48: K48W; position 72: L72F; position 81: R81D. The amino acid sequence of the wild-type human IL-2 is shown as SEQ ID NO: 1.

Most preferably, the above mutant is one of the mutational schemes in the table below:

| mutant | mutation site |
|---|---|
| IL-2 | — |
| G438P1 | K8R/L18M/L19S/K48W |
| G438P8 | K8R/K48W/L72F/R81D |
| G438P12 | K8R/L18M/L19S/K48R |
| G438P20 | K9R/L18M/L19S/K48R |
| G438P21 | lack of the first amino acid A, K9R/L18M/L19S/K48R |
| G438P22 | K8R/L18M/L19S/K49R |

Third, the inventors also considered the need for IL-2 mutants to reduce the bias toward IL-2Rα, maintain or enhance the bias toward IL-2Rβ and IL-2Rγ. So the mutation is that one, two or more amino acids at position 1, 18, 19, 27, 35, 38, 41, 42, 43, 45, 54, 64, 65, 72, 78, 79, 80, 81, 82, 83, 87, 92, 97 from the N-terminus of wild-type human IL-2 is substituted, such as G438P8.

IL-2 acts through IL-2 receptor (IL-2R), which consists of three subunits, IL-2Rα(CD25), IL-2Rβ(CD122), and IL-2Rγ(CD132). The three subunits form three receptor forms. The high affinity receptor contains all three subunits IL-2Rα, IL-2Rβ and IL-2Rγ. The moderate affinity receptor contains IL-2Rβ and IL-2Rγ. The low affinity receptor contains IL-2Rα. IL-2Rα is highly expressed on treg cells; IL-2 Rβ is expressed on CD8+T cells, NK cells, treg cells; IL-2 Rγ is expressed on all the immune cells. IL-2 mediates multiple effects in the immune response by binding to receptors on different cells. On one hand, as immune system stimulator, IL-2 can stimulate the proliferation and differentiation of T cells, induce the generation of cytotoxic T lymphocytes, promote the proliferation and differentiation of B cells and immunoglobulin synthesis, and stimulate the generation and activation of NK cells. IL-2 has been approved as immune agents for the treatment of cancer and chronic virus infection. On the other hand, IL-2 can promote the activation and proliferation of the immunosuppressive CD4+CD25+ regulatory T cells (Treg cells), resulting in immunosuppression. A large number of studies have proposed that by changing the bias of IL-2 to IL-2Rα, the toxic side effects of IL-2 as an immune system stimulator can be reduced and its efficacy can be improved. There have been many reports of changing the bias toward IL-2 receptor by site mutation, one skilled in the art can select IL-2 with other sites mutations for polyethylene glycol modification, but not limited to the specific mutations mentioned above.

A further object of the present invention is to provide polyethylene glycol modified human IL-2 mutants, using polyethylene glycol succinimidyl succinate as PEG modifier, with an average of 5-8 PEG molecules conjugated to one human IL-2 molecule; the human IL-2 mutant is one of the different mutants mentioned above.

Another object of the present invention is to provide application of polyethylene glycol modified human IL-2 or mutant in the preparation of drug for the treatment of neoplastic disease. Neoplastic disease includes but not limited to: squamous cell carcinoma, melanoma, colon cancer, breast cancer, ovarian cancer, prostate cancer, gastric cancer, liver cancer, small cell lung cancer, non-small cell lung cancer, thyroid cancer, kidney cancer and bile duct cancer, brain cancer, cervical cancer, maxillary sinus cancer, bladder cancer, esophageal cancer, Hodgkin's disease, and adrenal cortical carcinoma.

A further object of the present invention is to provide composition for the treatment of neoplastic disease, comprising polyethylene glycol modified human IL-2, human IL-2 mutant, or polyethylene glycol modified human IL-2 mutant, also comprising anti-HER2 antibody, anti-PD-1 antibody, anti-PD-L1 antibody or anti-CD26 antibody.

The anti-HER2 antibodies include Herceptin, Perjeta and Kadcyla from Roche.

The anti-PD-1 antibodies include Opidivo from Bristol-Myers Squibb, Keytruda from Merck Sharp & Dohme, Toripalimab from Junshi Bio, Sintilizumab from Cinda Bio, camrelizumab from Hendrik Pharma, tisllizumab from Baigene, and the like.

The anti-PD-L1 antibodies include Tecentriq from Roche, Imfinzi from Astrazeneca, Bavencio from Merck, and the like.

The anti-CD26 antibody can be anti-CD26 antibody such as YS110 (patent CN200680034937.4), or patent CN202111245489.5.

The combination is used for the treatment of neoplastic disease, including but not limited to: squamous cell carcinoma, melanoma, colon cancer, breast cancer, ovarian cancer, prostate cancer, gastric cancer, liver cancer, small cell lung cancer and non-small cell lung cancer, thyroid cancer, kidney cancer and bile duct cancer, brain cancer, cervical cancer, maxillary sinus cancer, bladder cancer, esophageal cancer, Hodgkin's disease, and adrenal cortical carcinoma.

The invention has the following technical effects:

1, Polyethylene glycol is linked to IL-2 by an amide bond through the reaction of polyethylene glycol succinimidyl succinate with the ε-amino group of lysine side chain in IL-2 and its mutants. Since the receptor binding site is completely masked, the modified PEG-IL-2 molecule was inactive. At the same time, unlike other modifiers, there is an ester bond in the conjugate of polyethylene glycol succinimidyl succinate modified protein or polypeptide. The ester bond is prone to hydrolysis due to its inherent instability, leading to the loss of PEG chain. In the past, this property was generally regarded as easy shedding of the modifier and causing instability of the modified molecule, so the PEG modifier was gradually replaced by other PEG modifiers that are difficult to shed, such as polyethylene glycol succinimidyl propionate, etc. However, in the present invention, the hydrolytic property makes IL-2 have the property of gradually releasing drug activity in vivo, and extremely excellent drug effect can be obtained using PEG modifier with specific molecular weight and specific PEGylation degree.

2, IL-2 do not contain adjacent lysines by mutating the lysine, which limits the position of PEG coupling, greatly reduce the production of isomers with different PEG modified site, facilitate the uniformity control of PEG modification product. On this basis, through further mutation site design and screening, we obtained mutant with significant reduced a receptor affinity, maintained β receptor affinity, which has significantly better proliferation activity against CTLL-2 cells than wild-type IL-2.

3. In practice, the activity of PEG-IL-2 will be slowly released with the gradual shedding of PEG. In the present invention, through the optimization and screening of different molecular weights and different binding numbers of PEG, not only the low bioavailability of high-modified IL-2 is avoided, but also the high toxic side effects of low-modified IL-2 is avoided. The PEGylated IL-2 of the present invention effectively achieves the dynamic balance of the bioavailability and safety. On the other hand, it also achieves stable blood drug concentration and better availability in vivo through different dosage and frequency of administration, so as to achieve better efficacy in vivo.

### BRIEF DESCRIPTION OF THE DRAWINGS

FIG. 1. CTLL 2 cell proliferation by IL-2 mutant stimulating

FIG. 2. Activity of PEG-SS modified different mutants to stimulate CTLL-2 cells proliferation under alkaline conditions

FIG. 3. Determination of phosphorylated STAT5 level of CTLL-2 stimulated by PEG-SS (with different structures and molecular weight) modified IL-2 mutants

FIG. 4. Determination of phosphorylated STAT5 level of CTLL-2 stimulated by PEG-SS modified different IL-2 mutants

FIG. 5. In vivo pharmacokinetics comparison between mPEG-SS-5k-GP8-high modification and the original protein GP8

FIG. 6. Efficacy evaluation of different PEG-SS (linear or branch) modified IL-2 in BALB/c mice bearing subcutaneous CT26.WT murine colon cancer cell xenografts. FIG. 6a shows the tumor growth curves of animals in each group, FIG. 6b shows tumor weight of animals in each group, FIG. 6c shows body weight change of animals in each group.

FIG. 7. Efficacy evaluation of different IL-2 mutants modified with linear chain PEG-SS in C57BL/6 mice bearing subcutaneous B16-F10 murine melanoma cell xenografts. FIG. 7a shows the tumor growth curves of animals in each group, and FIG. 7b shows tumor weight of animals in each group.

FIG. 8. Efficacy evaluation of IL-2 mutants modified with PEG-SS (with different molecular weight and different degree of PEGylation) in B16-F10 murine melanoma tumor model. FIG. 8a shows the tumor growth curves of animals in each group. FIG. 8b shows the tumor weight of animals in each group on day 17 after cell inoculation. FIG. 8c shows body weight change of animals in each group.

FIG. 9. Efficacy evaluation of IL-2 mutants modified with PEG-SS (with different molecular weights and different degree of PEGylation) in CT26.WT murine colon tumor model. FIG. 9a shows the tumor growth curves of animals in each group. FIG. 9b shows tumor weight of animals in each group on day 22 after cell inoculation. FIG. 9c shows body weight change of animals in each group.

FIG. 10. Efficacy evaluation of IL-2 mutants modified with PEG-SS (with different molecular weights and different degree of PEGylation) in A375 human melanoma model. FIG. 10a. shows the tumor growth curves of animals in each group. FIG. 10b shows tumor weight of animals in each group on 45 days after inoculation. FIG. 10 c shows body weight change of animals in each group.

FIG. 11. Efficacy evaluation of different doses of PEG modified IL-2 mutants in A375 human melanoma model. FIG. 11a shows the tumor growth curves of animals in each group. FIG. 11b shows tumor weight of animals in each group. FIG. 11 c shows body weight change of animals in each group.

FIG. 12. Efficacy evaluation of different doses of PEG modified IL-2 mutants in A498 renal cancer model. FIG. 12a shows the tumor growth curves of animals in each group. FIG. 12b shows tumor weight of animals in each group. FIG. 12c shows body weight change of animals in each group.

### DETAILED DESCRIPTION OF THE INVENTION

### Definition

Interleukin-2: IL-2, can be recombinant or non-recombinant, and can be wild type or mutant. IL-2 can be expressed in bacteria (E. coli), mammalian cells (E. CHO), or yeast (E. Pichia pastoris). IL-2 derives from human or an animal, derives from human preferably. In a specific embodiment of the present application, the human IL-2 amino acid sequence is shown as SEQ ID NO: 1, and the mutant is obtained by substitution, insertion, or deletion of some amino acids based on this sequence. In the specific embodiment of polyethylene glycol modified IL-2, IL-2 as shown in SEQ ID NO: 1 and its mutant is used, but one skilled in the art is aware that specific embodiments are intended only for illustration but not to limit the scope of the present application. Polyethylene glycol modified IL-2 may also be selected from other reported human IL-2 sequences or their mutant, said mutant is obtained by mutating the amino acid at corresponding position of SEQ ID NO: 1, thus has no adjacent lysine, reduced IL-2Rα bias, enhanced IL-2Rβ bias, and enhanced IL-2Rγ bias.

Polyethylene glycol: PEG, usually formed by the polymerization of ethylene oxide, has branched, linear, and multi-arm forms. In general, those with molecular weight below 20,000 are referred as PEG and those with larger molecular weight are referred as PEO. Ordinary polyethylene glycol has hydroxyl group at each end. If one end is blocked with methyl group, methoxy polyethylene glycol (mPEG) is obtained.

Polyethylene glycol modifiers: PEG modifiers refer to PEG derivatives with functional groups, which are activated polyethylene glycol and can be used for protein and peptide modification. The polyethylene glycol modifier used in this application was purchased from ZonHon Biopharma Institute, INC. or Beijing Jiankai Technology Co., LTD. The actual molecular weight of PEG modifier can be 90%-110% of the labeled value, such as the actual molecular weight of PEG5K can be 4.5 kDa-5.5 kDa, the actual molecular weight of PEG20K can be 18 kDa-22 kDa. When the labeled value of PEG modifier is 5-20 kDa, the actual molecular weight is 4.5 kDa-22 kDa.

V-PEG-SC-20k used in examples refers to branched polyethylene glycol succinimidyl carbonate with molecular weight of 20 kDa. The PEG modifier is prepared with reference to patent CN200680029849.5, According to the patent, the PEG modifier reacts with the drug through a linker to form a conjugate. The shedding of PEG from the conjugate can achieve the purpose of sustained and controlled release of drug activity, which is consistent with the structure of NKTR-214 reported. Therefore, the applicant uses this PEG modifier to modify IL-2 as positive reference. The structure of V-PEG-SC-20k is shown as follows:

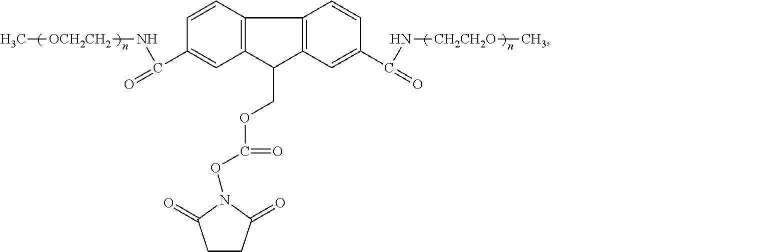

wherein n is an integer from 199 to 244.

V-PEG-SS-20k used in examples refers to branched polyethylene glycol succinimidyl succinate modifier with molecular weight of 20 kDa; The structure of V-PEG-SS-20k is shown as follows:

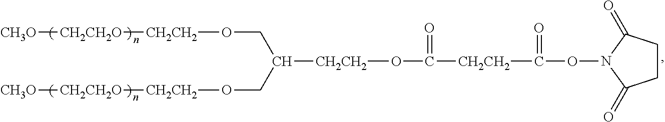

wherein n is an integer from 198 to 244.

mPEG-SS-20k/10k/5k used in examples refers to straight-chain polyethylene glycol succinimidyl succinate modifier with molecular weight of 20 kDa, 10 kDa, and 5 kDa, respectively;

The mPEG-SS-20k/10k/5k modifier structure is shown as follows:

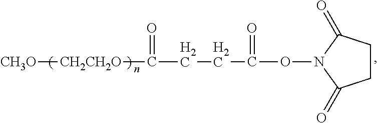

in mPEG-SS-20k: n is an integer from 403 to 494;
in mPEG-SS-10k: n is an integer from 199 to 244;
in mPEG-SS-5k: n is an integer from 97 to 119.

mPEG-SPA-5k used in examples refers to straight-chain polyethylene glycol succinimidyl propionate modifier with molecular weight of 5 kDa; with structure as follows:

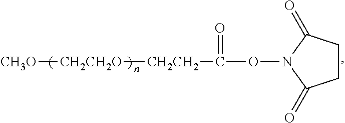

wherein n is an integer from 98 to 120.

### Example 1: IL-2 Mutant Design and Preparation

### 1, IL-2 Mutant Design

In the following table, IL-2 was wild-type human IL-2 with the amino acid sequence shown in SEQ ID NO: 1, and a mutant was obtained by substitution, insertion, or deletion of some amino acids based on this sequence. For Example, G438 was obtained by substitution of amino acids at position 8 and 48 based on SEQ ID NO: 1. K8R referred to the replacement of lysine at position 8 with arginine.

TABLE 1
Mutation site information Table

| Mutant | Mutation site |
|---|---|
| IL-2 | — |
| G438 | K8R/K48W |
| G493 | K8R/K43W/K48W/K54W/K64H/K97E |
| G495 | K8R/K43W/K48Y/K54W/K64H/K97T |
| G496 | K8R/K43W/K48W/K54W/K64H/K97R |
| G498 | K8R/K43W/K48W/K54W/K64H/K97F |
| G499 | K8R/K43W/K48Y/K54F/K64H/K97T |
| G500 | K8F/K43W/K48W/K54F/K64H/K97R |
| G438P1 | K8R/L18M/L19S/K48W |
| G438P2 | K8R/K35E/K48W/K43D |
| G438P3 | K8R/G27C/K48W/F78C |
| G438P4 | K8E/K35D/K43E |
| G438P5 | K8E/L19S/K35D |
| G438P6 | K8R/F42A/K43D/K48W |
| G438P7 | K8R/R38E/T41E/K48W |
| G438P8 | K8R/K48W/L72F/R81D |
| G438P9 | K8R/L18M/L19S/F42A/K48W |
| G438P10 | K8R/L18M/L19S/K48W/H79E/R81D |
| G438P12 | K8R/L18M/L19S/K48R |
| G438P13 | K8R/L18M/L19S/K49D |
| G438P14 | K8R/L18M/L19S/R38E/T41E/K48W |
| G438P15 | K8R/L18M/L19S/K48W/R38E/T41E/Y45R/L72F |
| G438P16 | K8R/L18M/L19S/K48W/R38E/T41E/P82A/S87D |
| G438P17 | K8R/L18M/L19S/K48W/R81D/P82T/R83E/S87D |
| G438P19 | lack of the first amino acid A, K8R/L18M/L19S/K48W/ |
| | A125S, IISTLT from position 128 mutated to SSQH |
| G438P20 | K9R/L18M/L19S/K48R |
| G438P21 | lack of the first amino acid A, K9R/L18M/L19S/K48R |
| G438P22 | K8R/L18M/L19S/K49R |
| G438P23 | lack of the first amino acid A, K8R/L18M/L19S/K49R |
| G438P25 | K8R/K48R/P65K/L72F/L80F/R81D/192F |
| G438P26 | K8R/K48R/L72F/R81D/R83E |

### 2. Protein Preparation

Conventional recombinant protein preparation methods were used and were not limited to the methods described in examples. Take the preparation of IL-2 mutant G438 as example:

Step 1: Based on the amino acid sequence of G438 (a sequence with K8R/K48W mutation based on wild-type IL-2 shown as SEQ ID NO:1), codon optimization was performed on Escherichia coli to obtain the coding DNA sequence as shown in SEQ ID NO:2. The DNA sequence was cloned into the pBV220 vector to form the recombinant plasmid pBV220-G438, and then the recombinant plasmid was transformed into Top10 E. coli to form the expression host TOP10-pBV220-G438.

Step 2: Top10-pBV220-G438 was inoculated into TB medium (500 ml medium, liquid volume was 20%), and cultured at 30° C. and 220 rpm shaking until the culture medium OD600 reached 1.00.1, and then the shaking speed was maintained and the culture temperature was raised to 42° C. and inducible expression was maintained for 4 h. After the inducible expression and centrifugation, collected bacterial precipitate.

Step 3: The bacterial precipitation was resuspended in 10 mM pH7.4 PBS to 100 g/L, and then lysed using a probe type ultrasonic cell breaker (working power 250w, working for 3 s and pause for 4 s, total crushing for 30 min). Centrifuge to collect cell lysate, discard the supernatant, collect the precipitate, which is the G438P8 inclusion body.

Step 4: The G438 inclusion body was resuspended to 50 g/L with PBS+1% TritonX100, mixed with a magnetic stirrer for more than 2 hours, and the precipitate was collected by centrifugation. Repeat this operation 3 times to obtain the G438 crude pure product.

Step 5: The crude pure G438 inclusion bodies were resuspended to 1 g/100 ml with denaturing solution (20 mM Tris, 8M urea, 5 mM DTT, pH10.5), denatured for more than 2 hours with stirring, centrifugated and collected the supernatant. The supernatant was purified with superdex 75.

Step 6: The purified G438 was diluted and renatured with renaturation buffer (20 mM Tris, 2M urea, 3 mM cysteine, 1 mM cystine, 0.01% SDS, pH8.0), the protein concentration in the renaturation system was not higher than 0.1 mg/ml, the renaturation time was more than 36h, and the renaturation temperature was 15° C.

Step 7: The renatured G438 was concentrated by ultrafiltration. The concentrated samples were dialyzed to remove urea and other reagents with pH7.4 PBS, resulting G438 pure products.

The amino acids of each mutant can be obtained from the sequence as shown in SEQ ID NO: 1 in combination with the mutation scheme listed in Table 1. Mutants for each of the above designs were prepared using similar methods as described above.

### Example 2: Receptor Affinity Assay for IL-2 Mutants

### 1. Experiment Method

The affinity between IL-2 mutants and their receptors was detected by Bio-Layer Interferometry (BLI).

### 1). Sample Preparation

Test sample solution: protein samples were diluted to 300 mm with 1×Kinetics buffer respectively, blended, and set aside.

Receptor solution: IL-2Rα(CD25), IL-2Rβ(CD122) and IL-2Rβ/γ receptor samples were diluted to 15-20 μg/mL with 1×Kinetics buffer, mixed and stored in a dark place.

### 2). Add the Sample

Samples were added to the sample wells according to the protocol design, and 200 μL of reagent or sample was added to each well.

### 2. The Experimental Results

Run the program and analyze the data with Fortebio Data Analysis 8.0 software, and calculate the affinity binding values as shown in the table below:

TABLE 2
Affinity binding values of IL-2 mutants to their receptors
IL-2Rα(CD25) and IL-2Rβ(CD122)

| | Affinity to | Affinity to |
|---|---|---|
| mutant | IL-2α K_{D} (M) | IL-2β K_{D} (M) |
| IL-2 | 8.65E−09 | 5.34E−08 |
| G438 | 1.34E−08 | 1.53E−08 |
| G493 | ND | ND |
| G495 | 5.21E−05 | 2.11E−07 |
| G496 | ND | ND |
| G498 | ND | ND |
| G499 | ND | ND |
| G500 | ND | ND |
| G438P1 | 1.79E−08 | 1.83E−08 |
| G438P2 | 6.89E−08 | 1.83E−08 |
| G438P3 | 2.64E−08 | 1.21E−08 |
| G438P4 | ND | ND |
| G438P5 | ND | ND |
| G438P6 | 1.19E−07 | 5.23E−08 |
| G438P7 | 1.78E−04 | 6.42E−08 |
| G438P8 | 1.31E−07 | 1.20E−08 |
| G438P9 | 3.47E−08 | 2.64E−08 |
| G438P10 | 3.61E−08 | 3.05E−08 |
| G438P12 | 8.48E−09 | 7.29E−09 |
| G438P13 | 7.51E−09 | 7.47E−09 |
| G438P14 | ND | 4.26E−05 |
| G438P15 | 1.70E−07 | 8.71E−08 |
| G438P16 | 3.53E−07 | 1.42E−07 |
| G438P17 | 9.49E−08 | 6.10E−08 |
| G438P19 | 3.86E−08 | 1.29E−08 |
| G438P20 | 1.60E−08 | 4.49E−08 |
| G438P21 | 1.24E−08 | 5.64E−08 |
| G438P22 | 1.19E−08 | 2.94E−08 |
| G438P23 | 2.21E−08 | 4.21E−08 |
| G438P25 | 3.91E−08 | 4.39E−08 |
| G438P26 | 1.03E−08 | 2.44E−08 |

Note:
ND: None Detected, cannot be detected

TABLE 3
Affinity binding values of IL-2 mutants to their receptor IL-2Rβ/γ

| mutant | IL-2 | G438P8 | G495 | G438P7 | G438P14 | G438P15 |
|---|---|---|---|---|---|---|
| Affinity to | 1.82E−08 | 3.27E−08 | 9.05E−08 | 3.54E−08 | 3.96E−08 | 1.36E−04 |
| IL-2Rβ/γ KD(M) | | | | | | |

### 3. Analysis of Results

According to the results of receptor affinity assay, the mutants G493, G496, G498, G499, G500, G438P4 and G438P5 had significantly reduced or no binding to the two receptors, which may be caused by the great changes in protein structure after mutation. G495, G438P6, G438P7, G438P8, G438P14, G438P15 showed IL-2β binding bias. G495, G438P7, G438P8, G438P14 had little effect on the binding to IL-2Rβ/γ, while G438P15 had significantly decreased binding to IL-2Rβ/γ. The mutants that retained receptor binding ability in vitro were selected to further test the proliferation activity on CTLL-2 cells in vitro to evaluate their biological activity.

### Example 3: The Activity of IL-2 and its Mutants to Stimulate CTLL-2 Cells Proliferation

### I. Experimental Methods

CTLL-2 is mouse derived cell line, and the in vitro biological activities of IL-2, IL-2 mutants and modified IL-2 can be evaluated by detecting the proliferation rate of CTLL-2 cells at different concentration. The experimental method was CTLL-2/MTT colorimetric assay according to the 2020 edition of Chinese Pharmacopoeia Four General Rules 3524 “Human interleukin-2 biological activity assay”.

### 1. Preparation of Test Solution

RPMI 1640 culture medium: One bag of RPMI 1640 medium powder (specification: IL) was dissolved in water and diluted to 1000 ml, then 2.1 g sodium bicarbonate was added and dissolved, mixed, filtered, and stored at 4° C.

Basal culture medium: add 10 ml new born bovine serum (FBS) to 90 ml RPMI 1640 culture. Stored at 4° C.

Complete culture medium: human IL-2 was added to 100 ml of basal culture medium to a final concentration of 400-800 IU/ml. Stored at 4° C.

PBS: 100 ml of 10×PBS was aspirated and diluted to 1000 ml by adding water sterilized at 121° C. for 20 minutes.

MTT solution: 0.1 g MTT was dissolved with PBS and diluted to 20 ml. Bacteria were removed by filtration through a 0.22 μm filter membrane. Stored at 4° C. in the dark.

Lysis solution: 15% sodium dodecyl sulfate solution, which should not be used for more than 12 months.

### 2. Sample Preparation

The test solution with known protein content was diluted to suitable starting concentration. 2-fold serial dilutions were made for a total of eight dilutions, each dilution was added to two wells in 96-well cell culture plates. 50 μl solution was left in each well, and excess solution in the well was discarded. The above operations were performed under sterile conditions.

### 3. Cell Culture

CTLL-2 cells were cultured with complete culture medium at 37° C. in 5% carbon dioxide to sufficient amount. CTIL-2 cells were collected by centrifugation, washed three times with RPMI 1640 medium, resuspended in basal culture medium to prepare cell suspension containing 6.0×10⁵ cells per ml, and stored at 37° C. in 5% carbon dioxide for use. In the 96-well cell culture plate containing wild type and mutant samples, 500 cell suspension was added to each well, and the cells were cultured at 37° C. in 5% carbon dioxide for 18-24 hours. Then 20 μl of MTT solution was added to each well, and after 4-6 hours of incubation at 37° C. in 5% carbon dioxide, 150 μl of lysis solution was added to each well and kept at 37° C. in 5% carbon dioxide for 18 to 24 hours. All the above procedures were performed under sterile conditions. The liquid in the cell plate was mixed well and put into a microplate reader. The absorbance was measured at 570 nm wavelength with the reference wavelength 630 nm, and the measurement results were recorded. The sample concentration (ng/ml) was used as the abscissa, average OD570 detection value was used as the ordinate, ELISACalc software was used to fit the four-parameter response curve of the test sample.

$$Specific activity ( IU / mg ) = biological activity ( IU / ml ) / 20 ( ng / ml ) \times {10}^{6} .$$

### II. Experimental Results

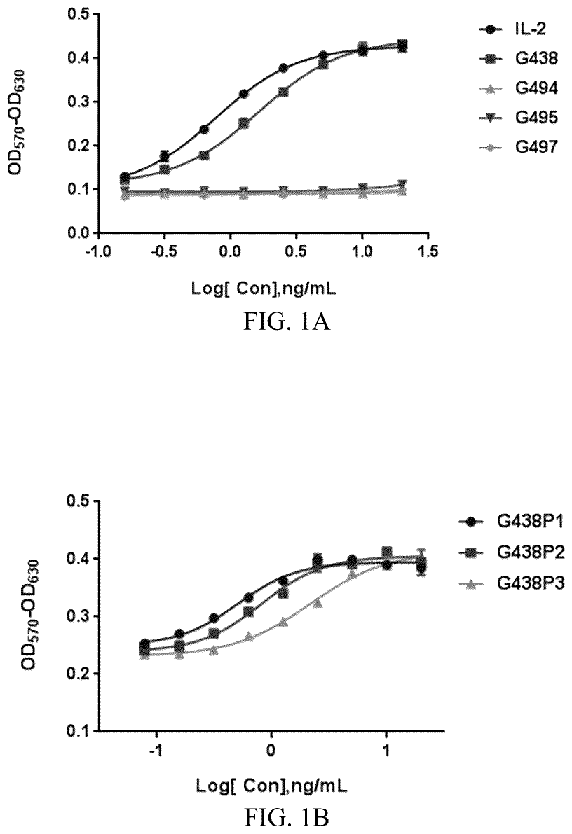

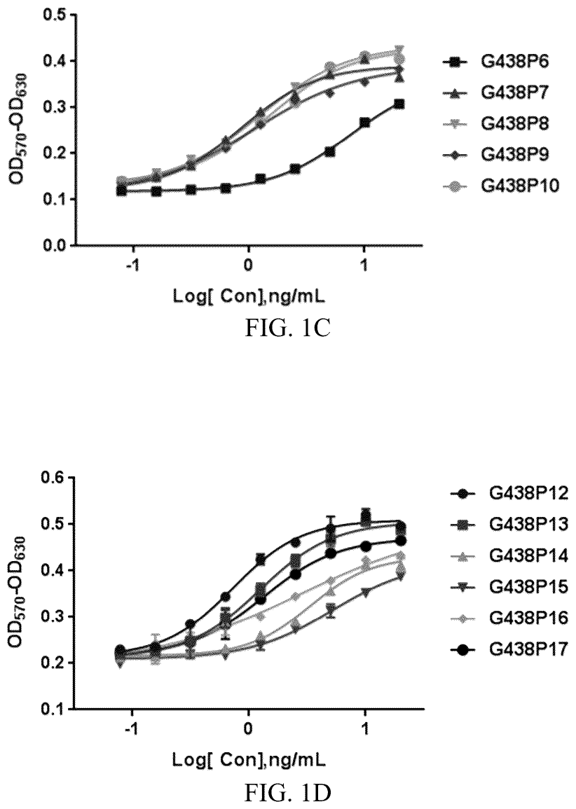

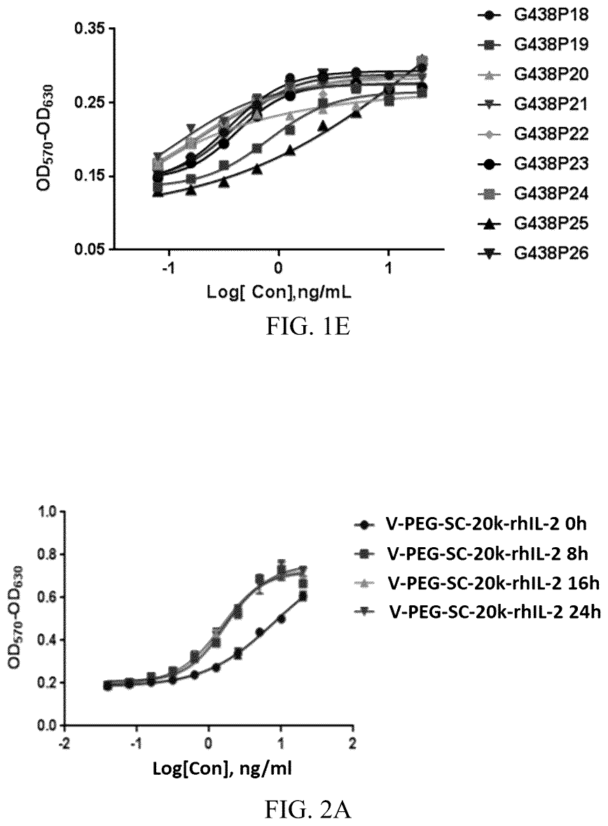

The test results were shown in FIG. 1 and the following table:

TABLE 3
Activity of IL-2 mutant to stimulate CTLL-2 cell proliferation

| | Specific | Relative |
|---|---|---|
| | activity | percentage |
| mutant | 10⁷ IU/mg | of activity |
| IL-2 | 1.12 | 100% |
| G438 | 0.79 | 70% |
| G495 | ND | ND |
| G438P1 | 1.55 | 138% |
| G438P2 | 0.15 | 13% |
| G438P3 | 0.04 | 4% |
| G438P6 | 0.06 | 5% |
| G438P7 | 0.58 | 52% |
| G438P8 | 1.30 | 116% |
| G438P9 | 0.49 | 44% |
| G438P10 | 0.66 | 59% |
| G438P12 | 1.69 | 150% |
| G438P13 | 0.96 | 86% |
| G438P14 | 0.24 | 21% |
| G438P15 | 0.12 | 11% |
| G438P16 | 0.39 | 35% |
| G438P17 | 0.76 | 68% |
| G438P19 | 0.45 | 40% |
| G438P20 | 1.66 | 148% |
| G438P21 | 2.69 | 240% |
| G438P22 | 2.12 | 189% |
| G438P23 | 1.13 | 101% |
| G438P25 | 0.17 | 15% |
| G438P26 | 1.30 | 116% |

ND: None Detected.
Test results were not obtained because proliferation curves could not be fitted

### 3. Analysis of Results

The newly constructed IL-2 mutants G438P1, G438P8, G438P12, G438P20, G438P21 and G438P22 had significantly higher activity to stimulate CTLL-2 cells proliferation than wild-type IL-2, and had the potential for clinical application.

Further modified mutants based on G438, such as: G438P1, G438P8, G438P12, G438P20, G438P21, G438P22 were abbreviated as GP1, GP8, GP12, GP20, GP21, GP22 respectively, and so on.

### Example 4: Preparation of PEG-Modified IL-2

### Example 4a: Preparation of Positive Reference V-PEG-SC-20k-rhIL-2

### 1. Modification Method

We selected V-PEG-SC-20k (purchased from Xiamen Sanobonge) published in patent CN200680029849.5 as modifier, and prepared PEG-IL-2 with the sustained release effect as positive reference.

The purified wild-type IL-2 protein was concentrated and replaced with modification buffer (100 mM disodium hydrogen phosphate-sodium dihydrogen phosphate, pH8.0) at the concentration of about 20 mg/mL. PEG was weighed, and the mass ratio of protein:PEG modifier was 1:20. The modification reaction was carried out at room temperature. The reaction was aborted by the addition of 1M glycine after 2 h of reaction.

### 2. The Purification Methods

Chromatography conditions: mobile phase A was 20 mm PB+2M NaCl (pH6.0), mobile phase B was 20 mM PB (pH6.0).

Loading: the above modified samples were diluted and loaded at 5 ml/min and combined to a hydrophobic chromatography column (purchased from GE, Phenyl PH).

Equilibration: after the loading, washed 15 to 20 column volumes with solution A.

Elution: eluted with 0-100% liquid B for 10 column volume, collected samples of V-PEG-SC-20k-rhIL-2.

The modified samples were purified and prepared according to the above conditions to obtain the required samples.

### 3. Purity Analysis

The SEC-HPLC results of the above PEG modified IL-2 and PEG modified IL-2 variant were shown in the table below:

TABLE 4
SEC-HPLC results of PEG modified IL-2 and PEG modified IL-2 variant

| | | Main peak | |
|---|---|---|---|
| | | retention time | |
| Number | Modified products | (min) | Purity |
| 1 | V-PEG-SC-20k-rhIL-2 | 9.6 | 98% |

SEC (size exclusion) chromatography is a chromatographic technique that separates the sample molecules according to their size. The PEG-IL-2 sample prepared in this Example was detected by SEC chromatography, and the results showed that the main peak of the sample was uniform, that was, the degree of PEGylation was uniform, which met the research requirements.

In each subsequent embodiment, unless otherwise specified, V-PEG-SC-20k-rhIL-2 of this preparation, or simply PEG-SC-20k-rhIL-2, was used as the positive reference.

### Example 4b: Preparation of Other PEG-Modified Samples Provided by the Invention

### 1. Modification Methods:

The purified wild type/mutant rhIL-2 samples were concentrated, replaced with modification buffer (100 mM disodium hydrogen phosphate-sodium dihydrogen phosphate, pH7.5) at the concentration of approximately 2-15 mg/mL, and PEG was weighed according to the modification reaction ratio shown in the table below (PEG types included but were not limited to: V-PEG-SS-20k, mPEG-SS-20k, m-PEG-SS-10k, mPEG-SS-5k, mPEG-SPA-5K), the modification reaction was carried out at room temperature, and the reaction was aborted by adding 1M glycine after 4 hours of reaction. The key reaction parameters of different products were shown in the following table:

TABLE 5
Process parameters of PEG-modified samples

| | | | Proprotein | Protein: PEG |
|---|---|---|---|---|
| | | | concentration | modifier(mass |
| Samples | Modifier | Proprotein | mg/ml | ratio) |
| V-PEG-SS-20k-GP1 | V-PEG-SS-20k | G438P1 | 10 | 1:25 |
| mPEG-SS-20k-GP1 | mPEG-SS-20k | G438P1 | 5 | 1:25 |
| V-PEG-SS-20k-GP8 | V-PEG-SS-20k | G438P8 | 10 | 1:20 |
| mPEG-SS-20k-GP8-mod | mPEG-SS-20k | G438P8 | 10 | 1:30 |
| erate modification | | | | |
| m-PEG-SS-10k-GP8 | m-PEG-SS-10k | G438P8 | 6 | 1:15 |
| mPEG-SS-5k-GP8 high | mPEG-SS-5k | G438P8 | 10 | 1:8 |
| modification | | | | |
| mPEG-SS-5k-GP8 | mPEG-SS-5k | G438P8 | 7.5 | 1:8 |
| moderate modification | | | | |
| mPEG-SS-5k-GP8 low | mPEG-SS-5k | G438P8 | 5 | 1:5 |
| modification | | | | |
| V-PEG-SS-20k-GP12 | V-PEG-SS-20k | G438P12 | 10 | 1:20 |
| mPEG-SS-20k-GP12 | mPEG-SS-20k | G438P12 | 10 | 1:30 |
| mPEG-SS-10k-GP12 | mPEG-SS-10k | G438P12 | 6 | 1:15 |
| mPEG-SS-5k-GP12 | mPEG-SS-5k | G438P12 | 5 | 1:10 |
| mPEG-SS-20k-GP13 | mPEG-SS-20k | G438P13 | 10 | 1:30 |
| mPEG-SS-20k-GP21 | mPEG-SS-20k | G438P21 | 10 | 1:30 |
| mPEG-SS-20k-GP22 | mPEG-SS-20k | G438P22 | 10 | 1:30 |
| mPEG-SS-20k-GP8- high | mPEG-SS-20k | G438P8 | 15 | 1:35 |
| modification | | | | |
| mPEG-SS-20k-GP8-low | mPEG-SS-20k | G438P8 | 2 | 1:20 |
| modification | | | | |
| mPEG-SPA-5k-rhIL-2 | mPEG-SPA-5k | rhIL-2 | 6 | 1:30 |

### 2. Purification Method

Chromatography conditions: mobile phase B was 20 mM NaAc+1M NaCl (pH4.0), mobile phase A was 20 mM NaAc (pH4.0).

Loading: The above modified samples were diluted, loaded at 5 mL/min, and combined to a cation-exchange chromatography column (purchased from GE, HPSP).

Equilibration: After the loading, washed with solution A for 15 to 20 column volumes.

Elution: eluted with 0-100% B solution for 10 column volumes, and samples of the main peak were collected.

The modified samples were purified and prepared according to the above conditions to obtain the required samples.

### 3. Purity Analysis

The SEC-HPLC results of PEG modified IL-2 and PEG modified IL2 variant were shown in the table below:

TABLE 6
SEC-HPLC results of PEG modified IL-2 and PEG modified IL2 variant

| | The retention | |
|---|---|---|
| | time of main | |
| Samples | peak(min) | Purity |
| V-PEG-SS-20k-GP1 | 9.7 | 100% |
| mPEG-SS-20k-GP1 | 9.2 | 97% |
| V-PEG-SS-20k-GP8 | 9.8 | 100% |
| mPEG-SS-20k-GP8- moderate | 9.5 | 95% |
| modification | | |
| m-PEG-SS-10k-GP8 | 9.9 | 96% |
| mPEG-SS-5k-GP8- low | 10.1 | 97% |
| modification | | |
| mPEG-SS-5k-GP8- moderate | 9.5 | 97% |
| modification | | |
| mPEG-SS-5k-GP8- high | 8.9 | 99% |
| modification | | |
| V-PEG-SS-20k-GP12 | 9.8 | 98% |
| mPEG-SS-20k-GP12 | 9.0 | 98% |
| mPEG-SS-10k-GP12 | 9.8 | 97% |
| mPEG-SS-5k-GP12 | 10.9 | 99% |
| mPEG-SS-20k-GP13 | 9.5 | 96% |
| mPEG-SS-20k-GP21 | 9.1 | 95% |
| mPEG-SS-20k-GP22 | 8.9 | 95% |
| mPEG-SS-20k-GP8- high | 9.1 | 95% |
| modification | | |
| mPEG-SS-20k-GP8-low | 10.0 | 96% |
| modification | | |
| mPEG-SPA-5k-rhIL-2 | 11.2 | 97% |

SEC (size exclusion) chromatography is a chromatographic technique that separates the sample molecules according to their size. The PEG-IL-2 sample prepared in this Example was detected by SEC chromatography, and the results showed that the main peak of the sample was uniform, that was, the degree of PEGylation was uniform, which met the research requirements. At the same time, taking GP8 as an Example, PEG-IL-2 samples with the same PEG and different PEGylation degree had significantly different retention time, indicating that the sample preparation process established in the invention can stably prepare samples with different PEGylation degree through the control of process parameters. The number of PEG binding for a specific sample is given in Example 5.

### Example 5: Determination of PEG Binding Number of PEG-Modified IL-2 (Hydrolysis Method)

### I. Experimental Methods

### 1. Preparation of Test Solution

1) Standard gradient PEG solution: take 5 μL, 10 μL, 20 μL, 30 μL, 40 μL, and 50 μL of V-PEG-SC-20k, V-PEG-SS-20k, mPEG-SS-20k, mPEG-SS-10k, and mPEG-SS-5k solution (2.5 mg/mL), respectively. The solution was added to water to make gradient solution with final volume of 100 μL, which was mixed to obtain standard gradient solution. 25 μL of 5×non-reducing Loding Buffer was added, mixed and set aside.

(2) The iodine dye solution: BaCl₂ 17.5 g, KI 6 g, and I₂ 3.9 g were accurately weighed and dissolved in 500 mL of double distilled water and stored away from light.

(3) Preparation of 10% perchloric acid solution: measured 100 ml of perchloric acid with measuring cylinder and slowly added it to 900 ml water, mix.

### 2. Gel Preparation

Preparation of 10% polyacrylamide gel: absorbed 1.6 mL of double distilled water, 1.8 mL of 30% polyacrylamide solution, 1.3 mL of 1.5 mol/L Tris-HCL pH8.8, 0.53 mL of 1% SDS solution, 0.033 mL of 10% ammonium persulfate solution and 0.033 mL of TEMED. Mixed well and made separation gel.

After the separation gel was solidified, mix 2.9 mL double distilled water, 0.9 mL of 30% polyacrylamide solution, 1.5 mL of 1.5 mol/L Tris-HCL pH6.8, 0.6 mL of 1% SDS solution, 0.047 mL of 10% ammonium persulfate solution and 0.047 mL TEMED evenly to make concentrated glue.

### 3. Sample Preparation

Hydrolysis: 200 μL activation buffer of 100 mM NaHCO₃ pH9.0 was added to 100 μL of test solution with known protein content and hydrolyzed in water bath at 37° C. for 24h (wherein mPEG-SPA-5k-rhIL-2 did not spontaneously hydrolyzate, the sample was treated at high temperature and incubated with trypsin). Samples were added to 5× non-reducing Loding Buffer, mixed, and set aside.

### 4. Electrophoresis Detection

Operating voltage: run at 80v for 30 min. When the bromophenol blue indicator moved to the bottom of the concentrated glue, the operation was changed to 120V until the bromophenol blue indicator moves to the edge of the bottom of the separation glue.

Iodine staining: at the end of electrophoresis, the glass plate was pry open, the film was labeled and placed in the staining box. The film was fixed with 10% perchloric acid solution for 10 min, and then the 10% perchloric acid was recovered. After washing with water for 3 times, the film was covered with iodine staining solution and stained for 2-3 min. Color can be developed in about 1 min, and then immediately decolorized with water.

### 5. Gel Imaging and Data Processing:

The decolorized gel was placed in the gel imager for gel imaging, and the Quantity One 4.4.0 software was used for quantification. The total amount of free PEG in the sample was obtained by linear regression of the PEG content of the standard and the gray level of the film spots. The results were calculated according to the following formula:

PEG binding number=moles of PEG/moles of protein

### II. Experimental Results

In vitro analysis results of PEG binding numbers of various modified products were shown in table below.

TABLE 7
In vitro analysis results of PEG binding number of modified products

| | PEG | | PEG |
|---|---|---|---|
| | binding | | binding |
| Sample | number | Sample | number |
| V-PEG-SC-20k-rhIL-2 | 6 | mPEG-SS-5k-GP8-low | 5 |
| | | modification | |
| V-PEG-SS-20k-GP1 | 7 | V-PEG-SS-20k-GP12 | 7 |
| mPEG-SS-20k-GP1 | 7 | mPEG-SS-20k-GP12 | 7 |
| V-PEG-SS-20k-GP8 | 7 | mPEG-SS-10k-GP12 | 7 |
| mPEG-SS-20k-GP8- high | 8 | mPEG-SS-5k-GP12 | 8 |
| modification | | | |
| mPEG-SS-20k-GP8-moderate | 7 | mPEG-SS-20k-GP13 | 7 |
| modification | | | |
| mPEG-SS-20k-GP8-low | 5 | mPEG-SS-20k-GP21 | 6 |
| modification | | | |
| mPEG-SS-10k-GP8 | 7 | mPEG-SS-20k-GP22 | 6 |
| mPEG-SS-5k-GP8- high | 7 | mPEG- SPA-5k-rhIL-2 | 9 |
| modification | | | |
| mPEG-SS-5k-GP8-moderate | 6 | | |
| modification | | | |

* The PEG coupling of each molecule with random modification was slightly different, so the test results were not integer values. The data in this table had been integrated and real test results were slightly floating, between+0.5 of the values described in the table. As shown in table 7, PEG binding number of mPEG-SS-5k-GP8-high modification was 7, the actual binding number was 6.5-7.5.

### Example 6: Determination of In Vitro Sustained Release Performance of PEG-Modified IL-2

### I. Experimental Methods

The pretreatment method was referred to Example 5, in which the hydrolysis operation part was as follows: 200 μL of activation buffer of 100 mM NaHCO₃ pH9.0 was added to 6.6 μL of test solution with known protein content, hydrolyzed in water bath at 37° C. for 24h. At different time points (such as 8 h, 16 h, and 24h after the beginning of hydrolysis), 100 μL hydrolyzed sample was taken and diluted, added 5× non-reducing Loading Buffer, mixed for use.

2. The Sample Detection and Calculation Methods were the Same as Example 5.

### II. Experimental Results

In vitro PEG sustained-release performance analysis results of a variety of PEGylated products were shown in table below.

TABLE 8
In vitro PEG sustained-release performance analysis
results of a variety of PEGylated products

| | PEG | Release time/The number | | |
|---|---|---|---|---|
| | binding | of PEGs released | | |
| Samples | number | 8 h | 16 h | 24 h |
| V-PEG-SC-20k-rhIL-2 | 6 | 4.8 | 5.3 | 5.9 |
| V-PEG-SS-20k-GP1 | 7 | 4.5 | 4.8 | 6.5 |
| mPEG-SS-20k-GP1 | 7 | 5.3 | 6.0 | 6.6 |
| V-PEG-SS-20k-GP8 | 7 | 3.8 | 5.1 | 6.3 |
| mPEG-SS-20k-GP8-high | 8 | 6.6 | 6.8 | 8.0 |
| modification | | | | |
| mPEG-SS-20k-GP8 | 7 | 4.9 | 5.6 | 6.0 |
| moderate modification | | | | |
| mPEG-SS-20k-GP8-low | 5 | 2.3 | 3.1 | 4.2 |
| modification | | | | |
| mPEG-SS-10k-GP8 | 7 | 2.3 | 5.1 | 6.7 |
| mPEG-SS-5k-GP8 high | 7 | 2.4 | 4.6 | 6.5 |
| modification | | | | |
| mPEG-SS-5k-GP8 | 6 | 2.9 | 5.1 | 5.9 |
| moderate modification | | | | |
| mPEG-SS-5k-GP8 low | 5 | 3.5 | 4.5 | 4.9 |
| modification | | | | |
| V-PEG-SS-20k-GP12 | 7 | 2.6 | 4.1 | 6.1 |
| mPEG-SS-20k-GP12 | 7 | 3.3 | 4.8 | 6.4 |
| mPEG-SS-10k-GP12 | 7 | 5.1 | 5.7 | 6.5 |
| mPEG-SS-5k-GP12 | 8 | 1.8 | 6.7 | 7.8 |
| mPEG-SS-20k-GP13 | 7 | 5.1 | 5.3 | 6.8 |
| mPEG-SS-20k-GP21 | 6 | 4.3 | 5.1 | 5.6 |
| mPEG-SS-20k-GP22 | 6 | 3.9 | 5.0 | 5.7 |
| mPEG-SPA-5k-rhIL-2 | 9 | 0 | 0 | 0 |

* PEG shedding was slightly different between molecules at different time points, so the results were not integer values

### 3. Analysis of Results

This Example showed results in vitro at 37° C. in 10 mM NaHCO₃ pH9.0 activation buffer: IL-2 or its mutants modified with a variety of PEG-SS with different structures (straight chain or branched chain) and different molecular weight (5K, 10K, 20K) showed significant in vitro sustained release performance, and the positive reference also had ideal in vitro sustained release performance. However, other conventional PEG types with non-PEG-SS structure, such as SPA-PEG, PEG shedding cannot be achieved and cannot achieve the technical effect of sustained release without the introduction of additional groups (such as fluorene ring structure of the positive reference). Molecules with different molecular weight and different PEGylation degree showed different release rate in vitro, but whether the released active protein structure was effective still needed to be further evaluated by in vivo and in vitro activity.

### Example 7: Assay of the Activity of PEG-Modified IL-2 to Stimulate Proliferation of CTLL-2 Cells

### I. Experimental Methods

Due to the masking effect of PEG modification on protein active center, in vitro biological activities of modified protein will be reduced, so the samples needed to be pre-activated, the proliferation rate of CTLL-2 cells in response to different concentration of pre-activated sample could be detected to evaluate the biological activity of the modified products in the activated state in vitro.

### 1. Sample Preparation

Sample activation: 100 μL of the test solution with known protein content was added to 200 μl of 100 mM NaHCO₃ pH9.0 or 300 μL of 100% human serum, incubated in water bath at 37° C. for a certain time, and 100 μl of hydrolyzed sample was taken at regular intervals and mixed. Samples will be diluted to suitable initial concentration. 2-fold serial dilutions were made for a total of eight dilutions, each dilution was added to two wells in 96-well cell culture plates. 50 μl solution was left in each well, and excess solution in the well was discarded. The above operations were performed under sterile conditions.

### 2. The Test Solution Preparation, Cell Culture was the Same as Example 3.

### II. Experimental Results

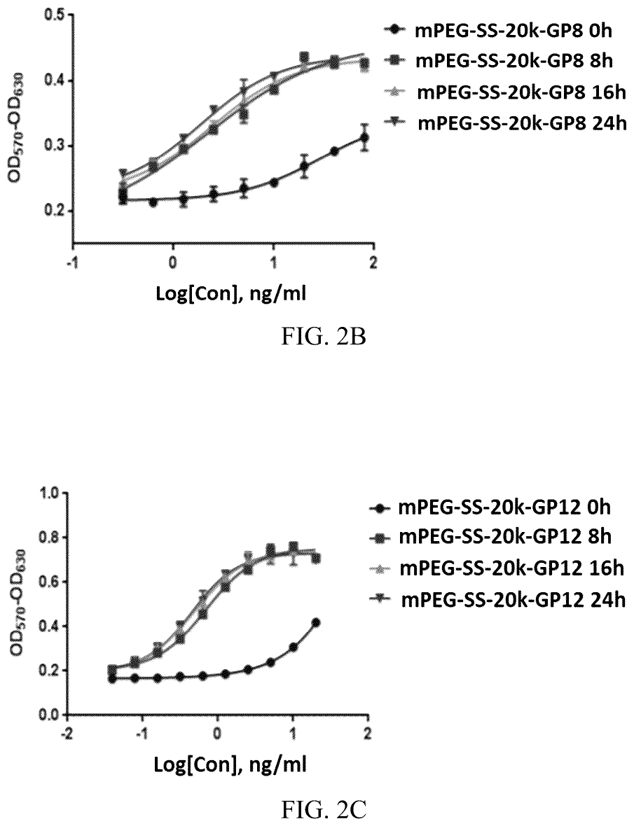

1. After being activated in 100 mM NaHCO₃ pH9.0, the activity of different polyethylene glycol modified products to stimulate the proliferation of CTLL-2 cells were shown in FIG. 2 and the table below (relative biological activity was calculated based on the activity value of the original protein used for PEG modification at 100% basis).

TABLE 9-1

| | | Relative |
|---|---|---|
| Sample | Activation time h | biological activity |
| V-PEG-SC-20k-rhIL-2 | 0 | 0% |
| | 8 | 56.6% |
| | 16 | 64.5% |
| | 24 | 61.9% |
| mPEG-SS-20k-GP12 | 0 | 0% |
| | 8 | 67.6% |
| | 16 | 92.9% |
| | 24 | 97.1% |
| mPEG-SS-20k-GP8 | 0 | 0% |
| | 8 | 63.4% |
| moderate modification | 16 | 93.6% |
| | 24 | 94.5% |

TABLE 9-2

| | | Relative |
|---|---|---|
| Sample | Activation time h | biological activity |
| mPEG-SS-20k-GP8 | 0 | 0% |
| high modification | 1 | 4.5% |
| | 2 | 7.3% |
| | 5 | 17.4% |
| | 9 | 32.4% |
| mPEG-SS-5k-GP8 | 0 | 0% |
| moderate modification | 1 | 28.7% |
| | 2 | 37.7% |
| | 5 | 60.3% |
| | 9 | 90.0% |
| mPEG-SS-5k-GP8 | 0 | 10% |
| low modification | 1 | 31.0% |
| | 2 | 49.5% |
| | 5 | 58.5% |
| | 9 | 68.8% |

### 3. Analysis of Results

The table 9 showed consistent with the reference substance V-PEG-SC-20k-rhIL-2, when the PEG-SS modified products was not activated (0 h after activation), the receptor binding site on the surface was completely masked by PEG. Therefore, it was not biologically active, which meant that the products was biologically inactive (undetectable) without PEG shedding. Both of them showed significant biological activities in promoting the proliferation of CTLL-2 cells after activation in alkaline conditions in vitro for different times. In addition, the bioactivity of PEG-SS modified products gradually recovered and increased with the prolongation of activation time. The biological activities of mPEG-SS-20k-GP8 and mPEG-SS-20k-GP12 with specific PEGylation degree were higher than that of V-PEG-SC-20k-rhIL-2. When the PEG shedding behavior was similar, better activity release can be achieved by adjusting the PEGylation degree.

In addition, PEG with different molecular weight had different active release effects with different PEGylation degree, which also formed the basis for the adjustable sustained release performance of PEG-SS modified IL-2. That is, we can explore the optimal release curve of PEG-IL2 in vivo by selecting the PEG-SS with different molecular weight and controlling the PEG binding number, so as to achieve the optimal bioavailability of the effector molecule in vivo, and obtain candidate molecules with better efficacy than existing technology.

### Example 8: Determination of the Activity of—IL-2 Modified with Different PEG to Stimulate pSTAT5 on CTLL-2 Cells

### I. Experimental Methods

CTLL 2 cells were cultured with complete culture medium (RPMI 1640 medium+2 mM L-glutamine+1 mM sodium pyruvate+10% fetal bovine serum+10% T-STIM, added with concanavalin A) at 37° C. and 5% CO₂ to the density of 2×10⁵/mL, washed once with PBSA (PBS, pH7.2, 1% BSA), adjusted the cell density to 1×10⁶ cells/mL, and then divided into flow tubes, 500 μL per tube. Different concentration of PEG-modified IL-2 prepared with basal medium (RPMI 1640+2 mM L-glutamine+1 mM sodium pyruvate+10% fetal bovine serum) were added. After incubation at room temperature for 20 min, paraformaldehyde was immediately added to final concentration of 1.5%, vortexed and mixed, and incubated at room temperature for 10 min. In order to remove paraformaldehyde, added 1 mL of PBS, centrifuged at 1400 rpm for 5 min at 4° C. Cells were resuspended, 1 mL of 100% methanol precooled at 4° C. was added, vortexed and mixed, and incubated for 20 min at 4° C. Added 3 mL PBSA buffer, centrifugated at 1400 rpm for 5 min at 4° C., washed the cells twice. Added anti-Stat5 (pY694)-Alexa647 (BD, Cat #612599) and incubated for 30 min at room temperature in the dark. Washed twice with 3 ml PBSA, detected with BD Accuri™ C6 computer.

### II. Experimental Results

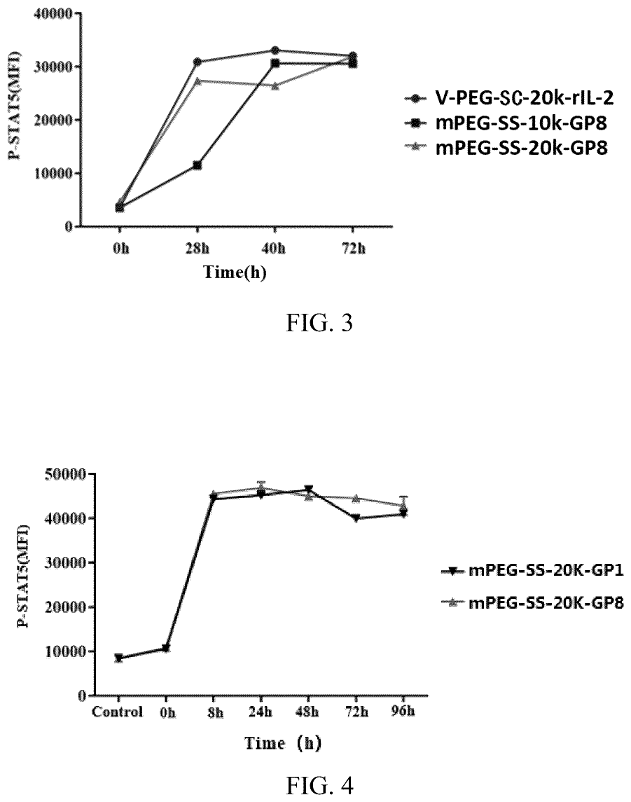

The results were shown in FIG. 3. pSTAT5 levels in vitro showed that the proliferation curves of activated phosphorylation levels of mPEG-SS-10k-GP8 after activation in serum were also significantly different from those of V-PEG-SC-20k-rhIL-2 and mPEG-SS-20k-GP8(mPEG-SS-20k-GP8-moderate modification as described above). The increase of phosphorylation level was relatively slow, presenting slowly increasing trend, and the activated phosphorylation level of the three samples was equivalent at 72h. It was highly consistent with the trend of PEG shedding to release active protein in vitro.

### 3. Analysis of Results

IL-2 exerts its biological function through JAK-STAT pathway when binding to any kind of IL-2R The JAK1 pathway by IL-2Rβ binding and the JAK3 pathway by IL-2Rγ binding results in the phosphorylation of key tyrosine residues on the β and γ subunits of IL-2R respectively, resulting in anchoring sites for other signaling molecules. Therefore, the biological activities of IL-2 and PEG-modified IL-2 in vitro could be evaluated by detecting the phosphorylation level of CTLL-2 cells treated with different concentrations of PEG-modified IL-2.

Therefore, the above experimental results showed that PEG-SS release rate in serum directly affected the pSTAT5 activation speed and the release speed of CTLL-2 proliferative activity. This structure of PEG had the ability to sustained-release IL-2 mutants in vivo to regulate the degree of immune activation.

### Example 9: Determination of the Activity of PEG-Modified Different IL-2 Mutants to Stimulate pSTAT5 on CTLL-2 Cells

### I. Experimental Methods

The same as Example 8

### II. Experimental Results

The detection results were shown in FIG. 4. The results of in vitro pSTAT5 levels showed that mPEG-SS-20k-GP1 and mPEG-SS-20k-GP8 (mPEG-SS-20k-GP8-moderate modification as described above) had similar trends in activated phosphorylation levels after activation in buffer, showing gradual activation over time, and maintained continuously after reaching the peak.

### 3. Analysis of Results

The above experimental results showed that different mutants of IL-2 modified by PEG-SS in the present invention (taking GP1 and GP8 as Examples) had similar trend of phosphorylation activation levels in vivo.

### Example 10: Comparison of Half-Life Prolongation and Activity Release of PEG Modified IL-2 Mutants In Vivo

### I. Experimental Methods

Experiment 1: SD rats were intravenously injected with 0.5 mg/kg, 0.1 mg/kg and 0.04 mg/kg of mPEG-SS-5k-GP8-high modification and 1 mg/kg of mutant GP8, respectively. Serum samples were collected before administration and 0.25 h, 1 h, 2 h, 4h, 8 h, 24h, 48 h, 72 h and 96 h after administration, and the levels of mPEG-SS-5k-GP8-high modification and mutant GP8 in serum were detected by two ELISA methods.

Experiment 2: SD rats were intravenously injected with 1 mg/kg of mPEG-SS-5k-GP8-high modification and positive reference V-PEG-SC-20k-rhIL-2. Serum samples were collected before administration and 0.25 h, 1 h, 2 h, 4 h, 8 h, 24 h, 48 h, 72 h and 96 h after administration, and CTLL-2 cell/MTT colorimetric method was used to measure the activity at each time point. The unit was IU/ml.

### 2. The Test Results

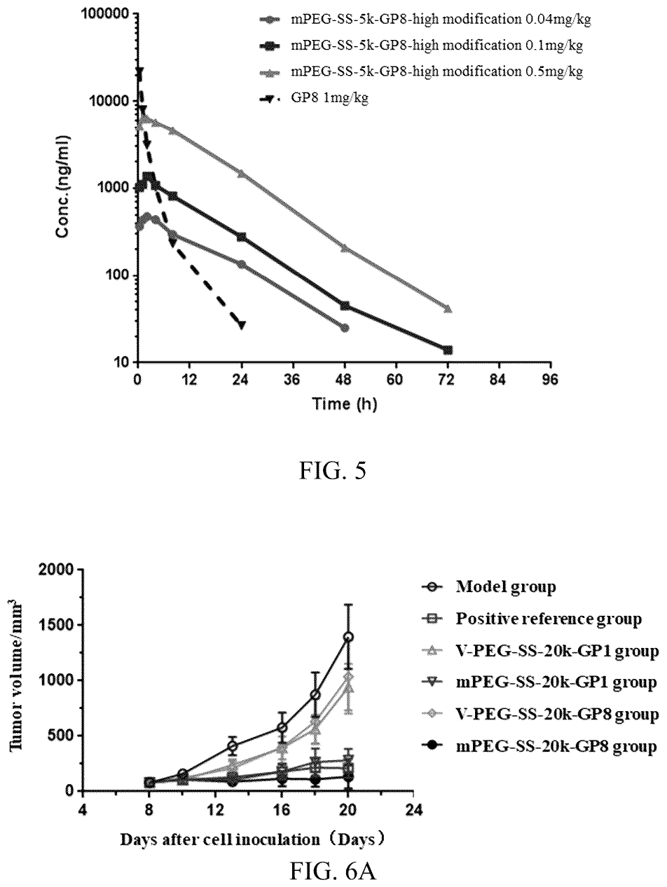

Experiment 1: as shown in FIG. 5, test results showed that, compared with mutant GP8 protein, mPEG-SS-5k-GP8-high modification can significantly extend the half-life.

Experiment 2: according to the test results, the drug time curve was drawn. The results showed that, the mPEG-SS-5k-GP8-high modification had higher AUC than the positive reference V-PEG-SC-20k-rhIL-2 at the same dosage and administration method (5683809h.IU/ml vs 2344730h.IU/ml)(calculated by GraphPad Prism 7.00). Therefore, mPEG-SS-5k-GP8-high modification had higher bioavailability than the positive reference V-PEG-SC-20k-rhIL-2.

### Example 11 Pharmacodynamic Evaluation of IL-2 Modified with Different Structures of PEG-SS (Straight Chain or Branched) in BALB/c Mice Bearing Subcutaneous CT26.WT Murine Colon Cancer Cell Xenografts

### I. Experimental Methods

CT26.WT cells were cultured in 1640 medium supplemented with 10% fetal bovine serum.

CT26.WT cells in the exponential growth phase were collected and resuspended in PBS to the appropriate concentration. CT26.WT cell suspension (5×10⁵ cells/0.1 ml) were mixed well and inoculated subcutaneously into the right flank of BALB/c mice. Each animal was inoculated with 0.1 ml. When the mean tumor volume reached about 100 mm³, the animals were divided into groups according to the tumor volume, and the first administration was started on the day of grouping (The first and second doses were administered respectively at the 9th day and the 16th day after cell inoculation in this experiment). The detailed dosage regimen and administration route were shown in the table below.

TABLE 10
Animal grouping and tumor cell inoculation information

| | | | | The volume | | |
|---|---|---|---|---|---|---|
| | | | | of drug | Administration | Administration |
| Groups | | N | Dosage | administered | route | cycle |
| 1 | Model group | 6 | — | — | — | — |
| 2 | Positive reference group | 6 | 2 mg/kg | 0.1 mL/10 g | i.v. | once a week, |
| | (V-PEG-SC-20k-rhIL-2) | | | | | for 2 weeks |
| 3 | V-PEG-SS-20k-GP1 | 6 | | | | |
| 4 | mPEG-SS-20k-GP1 | 6 | | | | |
| 5 | V-PEG-SS-20k-GP8 | 6 | | | | |
| 6 | mPEG-SS-20k-GP8 | 6 | | | | |

Note:
N: number of animals; s.c.: subcutaneous injection; i.v.: intravenous injection.
The day of cell inoculation was D 1.

General clinical observations: observations were conducted once daily at least during the pretest (the quarantine period) and trial period, including the tumor growth and the effects of treatment on health status of animals (The health status of animals including the death or near death, mental status, animal behaviour and other health status of tumor-bearing mice.

Body weight: Body weight was conducted twice or thrice a week.

Tumor volume: Tumor volume was conducted 2 to 3 times a week. The length and width of the tumor were measured by vernier caliper. Tumor volume: V=(length×width²)/2. Tumor Growth Inhibition value: TGI(%)=(1−T/C)×100%. It is generally accepted that T represents the relative tumor volume (the ratio of tumor volume of each measurement to tumor volume at the time of grouping) in the administration group and C represents the relative tumor volume (the ratio of tumor volume of each measurement to tumor volume at the time of grouping) in the model group. On the day of inoculation, the animals were grouped according to their weight. When calculating TGI, T and C represented the actual measured tumor volume of the administration group and the model group, respectively.

Tumor weight: Animals were euthanized at the end of the experiment, the tumor mass was dissected, rinsed with normal saline and blotted dry with filter paper, weighed and photographed. Tumor Growth Inhibition value: TGI(%)=(1−T_{TW}/C_{TW})×100% (T_{TW}: mean tumor weight of treatment group at the end of experiment, C_{TW}: mean tumor weight of control group at the end of experiment).

### II. Experimental Results

### 1, Efficacy:

The mean tumor volume of the model group was 1397.48±289.67 mm³ at the 20^{th} day after cell inoculation.

The mean tumor volume of mPEG-SS-20k-GP1 (2 mg/kg) and mPEG-SS-20k-GP8 (2 mg/kg)(mPEG-SS-20k-GP8-moderate modification) group on day 20 after cell inoculation was 282.31±103.02 mm³ and 133.93±105.69 mm³ respectively, there was significant difference (P<0.01) compared with model group. The tumor growth inhibition value TGI (%) was 79%, 92% respectively.

The mean tumor volume of V-PEG-SS-20k-GP1 (2 mg/kg), V-PEG-SS-20k-GP8 (2 mg/kg) on day 20 after cell inoculation was 942.44±209.66 mm³, 1037.67±332.97 mm³, there was no significant difference compared with model group. The tumor growth inhibition value TGI(%) was 26% and 29%, respectively.

The mean tumor volume of positive reference V-PEG-SC-20k-rhIL-2 (2 mg/kg) (For subsequent implementation, V-PEG-SC-20k-rhIL-2, or PEG-SC-20k-rhIL-2, was used as positive reference unless otherwise specified) was 211.23±86.02 mm³ on the day 20 after cell inoculation, which was significantly different from that of the model group (P<0.01), and the tumor growth inhibition value TGI (%) was 84%.

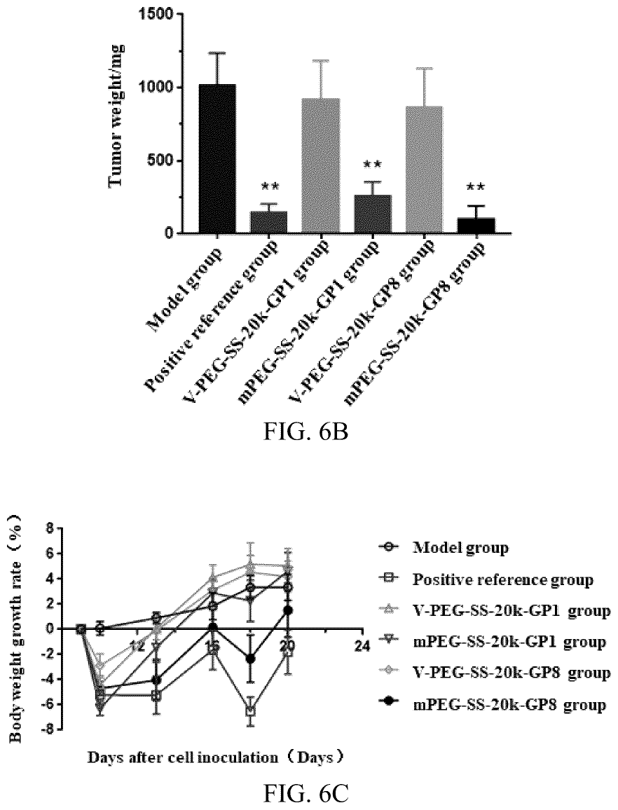

The results of tumor weight were similar to those of tumor volume. The experimental results were shown in the table below and FIG. 6:

TABLE 11
Tumor volume, relative tumor volume, TGI, T/C of animals in each group (Female, Mean ± SEM)

| | | on the day 20 after cell inoculation(day | | | | |
|---|---|---|---|---|---|---|
| | | 4 after the last dose) | | | | |
| | | | | | | P |
| | | | relative | | | value(vs |
| | N | tumor | tumor | TGI | T/C | model |
| Groups | (D 20/D 1) | volume(mm³) | volume | (%) | (%) | group) |
| Model group | 6/6 | 1,397.48 ± 289.67 | 16.69 ± 2.23 | — | — | — |
| Positive reference group | 6/6 | 211.23 ± 86.02 ** | 2.74 ± 1.07 | 84 | 16 | 0.003 |
| (V-PEG-SC-20k-rhIL-2) | | | | | | |
| V-PEG-SS-20k-GP1 | 6/6 | 942.44 ± 209.66 | 12.31 ± 3.06 | 26 | 74 | 0.232 |
| mPEG-SS-20k-GP1 | 6/6 | 282.31 ± 103.02 ** | 3.49 ± 1.22 | 79 | 21 | 0.005 |
| V-PEG-SS-20k-GP8 | 6/6 | 1,037.67 ± 332.97 | 11.83 ± 2.91 | 29 | 71 | 0.434 |
| mPEG-SS-20k-GP8 | 6/6 | 133.93 ± 105.69 ** | 1.41 ± 1.05 | 92 | 8 | 0.002 |

Note:
* P < 0.05,
** P < 0.01, vs model group.

TABLE 12
Tumor Weight, TGI, and T/C of animals in each group (Female, Mean ± SEM)

| | | on the day 20 after cell inoculation | | | |
|---|---|---|---|---|---|
| | | (day 4 after the last dose) | | | |
| | | | | | P value(vs |
| | N | | TGI | | model |
| Groups | (D20/D1) | Tumor Weight(mg) | (%) | T/C (%) | group) |
| Model group | 6/6 | 1,014.63 ± 243.16 | — | — | — |
| Positive reference group | 6/6 | 141.15 ± 63.48 ** | 86 | 14 | 0.0036 |
| (V-PEG-SC-20k-rhIL-2) | | | | | |
| V-PEG-SS-20k-GP1 | 6/6 | 916.50 ± 326.78 | 10 | 90 | 0.7831 |
| mPEG-SS-20k-GP1 | 6/6 | 257.65 ± 98.28 * | 75 | 25 | 0.0109 |
| V-PEG-SS-20k-GP8 | 6/6 | 864.90 ± 325.05 | 15 | 85 | 0.6744 |
| mPEG-SS-20k-GP8 | 6/6 | 98.23 ± 93.81 ** | 90 | 10 | 0.0035 |

Note:
* P < 0.05, ** P < 0.01, vs model group.

### 2. Safety:

The body weight loss of PEG-SS modified groups was less than 10% during the treatment period, and recovered later in the experiment, with no significant difference compared with the model group (D13, D16, D18, D20).

One animal in the positive reference group showed medium weight loss (10%<weight loss≤20%) on the second day after the second administration (D18). Although the weight of the positive reference group also recovered later in the experiment, the difference was significant compared with that of the model group.

TABLE 13
Body weight growth rate of animals in each group (Female, Mean ± SEM)

| | | Body weight growth rate(%) | | | | | |
|---|---|---|---|---|---|---|---|
| Groups | N | D9 | D10 | D13 | D16 | D18 | D20 |
| Model group | 6 | 0.00 ± 0.00 | 0.09 ± 0.54 | 0.90 ± 0.44 | 1.85 ± 1.08 | 3.34 ± 0.92 | 3.33 ± 1.05 |
| Positive reference | 6 | 0.00 ± 0.00 | −5.22 ± 0.35 ** | −5.24 ± 1.50 ** | −1.64 ± 1.59 | −6.53 ± 1.17 ** | −1.81 ± 1.75 * |
| group | | | | | | | |
| (V-PEG-SC-20k-rhIL- | | | | | | | |
| 2) | | | | | | | |
| V-PEG-SS-20k-GP1 | 6 | 0.00 ± 0.00 | −4.41 ± 0.71 ** | 0.08 ± 0.90 | 4.13 ± 1.00 | 5.19 ± 1.71 | 5.07 ± 1.36 |
| mPEG-SS-20k-GP1 | 6 | 0.00 ± 0.00 | −6.30 ± 0.55 ** | −1.45 ± 1.17 | 2.89 ± 1.06 | 2.27 ± 1.65 | 4.62 ± 1.50 |
| V-PEG-SS-20k-GP8 | 6 | 0.00 ± 0.00 | −2.88 ± 0.97 * | −0.11 ± 1.08 | 3.13 ± 0.96 | 4.55 ± 1.35 | 4.20 ± 1.25 |
| mPEG-SS-20k-GP8 | 6 | 0.00 ± 0.00 | −4.70 ± 0.55 ** | −4.04 ± 1.61 * | 0.14 ± 1.44 | −2.32 ± 1.87 * | 1.52 ± 2.15 |

Note:
* P < 0.05,
** P < 0.01, vs model group.

### 3. Analysis of Results

Linear PEG modified products mPEG-SS-20k-GP1, mPEG-SS-20k-GP8 significantly inhibited the tumor growth in CT26.WT colon cancer model at the dose of 2 mg/kg, and the TGI of those groups was similar to or higher than positive reference group. Branching PEG modified products V-PEG-SS-20k-GP1 and V-PEG-SS-20k-GP8 had no inhibitory effect in CT26.WT colon cancer model at the dose of 2 mg/kg.

Animals in each group were well tolerated at the dose of 2 mg/kg during this efficacy evaluation. In the effective groups, the body weight loss occurred both in the linear PEG-SS groups and the positive reference group in the middle period of treatment. But after the second dose and during the recovery period, the recovery of animal condition and body weight in the linear PEG-SS modified groups was significantly better than those of the positive reference group.

In this example, to the researchers' surprise, products modified with the linear mPEG-SS-20K and the branched V-PEG-SS-20k with the same molecular weight showed considerable differences in vivo test, and the branched PEG-SS modified product was much less effective than the linear PEG-SS modified products in vivo. As a result, the inventor confirmed the advantage to choose linear mPEG-SS to develop IL-2 protein drugs as tumor immune agonist.

### Example 12 Pharmacodynamic Evaluation of Linear PEG-SS Modified Different IL-2 Mutants in C57BL/6 Mice Bearing Subcutaneous B16-F10 Murine Melanoma Cell Xenografts

### I. Experimental Methods

B16-F10 cells were cultured in DMEM medium, containing 10% fetal bovine serum. B16-F10 cells in the exponential growth phase were collected and resuspended in PBS to the appropriate concentration. B16-F10 cells suspension (5×10⁵ cells/0.1 ml) were mixed well and inoculated subcutaneously into the right flank of C57BL/6 mice, and 0.1 ml was injected into each mouse. When the mean tumor volume reached about 100 mm³, the animals were divided into groups according to the tumor volume, and the first dose was started on the day of grouping (at the 7th day). The detailed dosage regimen and administration route were shown in the table below. General clinical observations was the same to Example 11.

TABLE 14
Animal Grouping and tumor cell inoculation information

| | | | | The volume | | |
|---|---|---|---|---|---|---|
| | | | | of drug | | Administration |
| Groups | | N | Dose | administered | Administration route | cycle |
| 1 | Model group | 6 | — | — | — | — |
| 2 | Positive reference group | 6 | 1 mg/kg | 0.1 mL/10 g | i.v. | once a week, |
| 3 | mPEG-SS-20k-GP1 | 6 | | | | for 1 week |
| 4 | mPEG-SS-20k-GP13 | 6 | | | | |
| 5 | mPEG-SS-20k-GP21 | 6 | | | | |
| 6 | mPEG-SS-20k-GP22 | 6 | | | | |

Note:
N: number of animals;
s.c: subcutaneous injection;
i.v.: intravenous injection.
The day of cell inoculation was D1.

### II. Experimental Results

### 1, Efficacy:

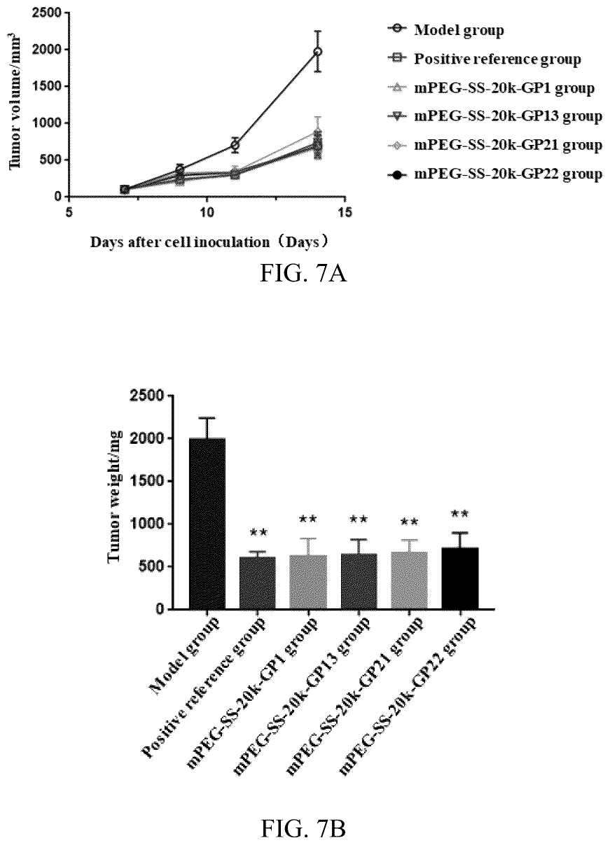

The experimental results were shown in the table below and FIG. 7.

TABLE 15
Tumor volume, relative tumor volume, TGI, T/C of animals in each group (Female,
Mean ± SEM)

| | | on the day 14 after cell inoculation(day 7 after the last dose) | | | | |
|---|---|---|---|---|---|---|
| | | | relative | | | |
| | N | tumor | tumor | TGI | T/C | P value(vs |
| Groups | (D14/D1) | volume(mm³) | volume | (%) | (%) | model group) |
| Model group | 4/6 | 1,978.59 ± 276.42 | 19.39 ± 2.13 | — | — | — |
| Positive reference | 4/6 | 700.65 ± 134.94** | 6.68 ± 1.08 | 66 | 34 | 0.0060 |
| group | | | | | | |
| mPEG-SS-20k-GP1 | 5/6 | 669.45 ± 152.85** | 6.93 ± 1.58 | 64 | 36 | 0.0032 |
| mPEG-SS-20k-GP13 | 5/6 | 739.71 ± 144.13** | 7.64 ± 1.44 | 61 | 39 | 0.0039 |
| mPEG-SS-20k-GP21 | 4/6 | 891.95 ± 200.49* | 9.26 ± 2.15 | 52 | 48 | 0.0190 |
| mPEG-SS-20k-GP22 | 5/6 | 691.60 ± 156.89** | 6.77 ± 1.15 | 65 | 35 | 0.0037 |

Note:
*P < 0.05,
**P < 0.01, vs model group.

TABLE 16
Tumor weight, TGI, and T/C of animals in each group (Female, Mean ± SEM)

| | | on the day 15 after cell inoculation(day 8 after the last dose) | | | |
|---|---|---|---|---|---|
| | N | | | T/C | P value(vs |
| Groups | (D14/D1) | Tumor weight(mg) | TGI (%) | (%) | model group) |
| model group | 4/6 | 1,988.58 ± 252.75 | | | |
| Positive reference group | 4/6 | 599.40 ± 77.88 ** | 70 | 30 | 0.0019 |
| mPEG-SS-20k-GP1 | 5/6 | 621.04 ± 215.19 ** | 69 | 31 | 0.0043 |
| mPEG-SS-20k-GP13 | 5/6 | 633.76 ± 189.93 ** | 68 | 32 | 0.0032 |
| mPEG-SS-20k-GP21 | 4/6 | 658.28 ± 157.26 ** | 67 | 33 | 0.0042 |
| mPEG-SS-20k-GP22 | 5/6 | 706.30 ± 194.54 ** | 64 | 36 | 0.0046 |

Note:
* P < 0.05, ** P < 0.01, vs model group.

### 2, Safety:

The body weight change rate of V-PEG-SC-20k-rhIL-2 group was significantly lower than that of the model group at D11 (all animals survived at D11). mPEG-SS-20k-GP1, mPEG-SS-20k-GP13, mPEG-SS-20k-GP21 and mPEG-SS-20k-GP22 were linear PEG-SS modified different mutants, and both had no significant difference in body weight loss compared with the model group at D1l. But V-PEG-SC-20k-rhIL-2 had significant difference in body weight loss compared with the model group at D11. To sum up, mPEG-SS-20k-GP1, mPEG-SS-20k-GP13, mPEG-SS-20k-GP21, mPEG-SS-20k-GP22 modified with linear PEG-SS had less effect on body weight than V-PEG-SC-20k-rhIL-2.

TABLE
Body weight change rate of animals in each group(Female, Mean ± SEM)

| | | Body weight change rate(%) | | | |
|---|---|---|---|---|---|
| Groups | N | D7 | D9 | D11 | D14 |
| Model group | 4-6 | 0.00 ± 0.00 | −0.30 ± 0.95 | 3.27 ± 1.40 | 10.55 ± 3.27 |
| Positive reference group | 4-6 | 0.00 ± 0.00 | −0.17 ± 1.06 | −2.64 ± 1.34* | 0.65 ± 0.72* |
| (V-PEG-SC-20k-rhIL-2) | | | | | |
| mPEG-SS-20k-GP1 | 5-6 | 0.00 ± 0.00 | 1.73 ± 0.75 | 4.25 ± 0.94^{ΔΔ} | −0.71 ± 4.92 |
| mPEG-SS-20k-GP13 | 5-6 | 0.00 ± 0.00 | −0.13 ± 0.85 | 2.36 ± 0.89^{Δ} | 4.38 ± 2.65 |
| mPEG-SS-20k-GP21 | 4-6 | 0.00 ± 0.00 | 2.31 ± 0.87 | 5.46 ± 1.04^{ΔΔ} | 6.86 ± 1.63 |
| mPEG-SS-20k-GP22 | 5-6 | 0.00 ± 0.00 | 2.27 ± 0.72 | 3.35 ± 1.02^{ΔΔ} | −0.67 ± 5.43 |

Note:
*P < 0.05, **P < 0.01, vs model group.
^{ΔΔ}P < 0.01, ^{Δ}P < 0.05 vs V-PEG-SC-20k-rhIL-2 group.
The death of animals occurred after D11 (all animals were alive at D11).

### 3. Analysis of Results

mPEG-SS-20k-GP1, mPEG-SS-20k-GP13, mPEG-SS-20k-GP21 and mPEG-SS-20k-GP22, which were linear PEG-SS modified different mutants, can inhibit tumor growth in B16-F10 murine melanoma model at the dose of 1 mg/kg, and the TGI of those groups was similar to positive reference group. The effect on the animal body weight of test sample groups (above samples) were less than that of 20k-V-PEG-SC-rhIL-2.

### Example 13: Pharmacodynamic Evaluation of Linear PEG-SS Modified ILr2 Mutants with Different PEGylation Degree in B16-F10 Murine Melanoma Tumor Model

### I. Experimental Methods

Experiment method and general clinical observations was the same as example 11. The detailed dosage regimen and administration route was shown in the table below.

TABLE 17
Animal grouping and tumor cell inoculation information

| | | | | The | | |
|---|---|---|---|---|---|---|
| | | | | volume of | | |
| | | | | drug | Administration | Administration |
| Groups | | N | Dose | administered | route | cycle |
| 1 | Model group | 5 | — | — | — | — |
| 2 | PEG-SC-20k-rhIL-2 | 6 | 2 mg/ | 0.1 mL/10 | i.v. | once a |
| 3 | mPEG-SS-20k-GP8(high | 6 | kg | g | | week, for |
| | modification) | | | | | 2 weeks |
| 4 | mPEG-SS-20k-GP8(moderate | 6 | | | | |
| | modification) | | | | | |
| 5 | mPEG-SS-5k-GP8(high | 6 | | | | |
| | modification) | | | | | |
| 6 | mPEG-SS-5k-GP8(moderate | 6 | | | | |
| | modification) | | | | | |
| 7 | mPEG-SS-5k-GP8(low | 6 | | | | |
| | modification) | | | | | |

Note:
N: number of animals;
s.c.: subcutaneous injection;
i.v.: intravenous injection.
The day of cell inoculation was D1.

### II. Experimental Results

### 1, Efficacy:

The mean tumor volume of the model group was 2614.97±372.77 mm³ on the day 17 after cell inoculation.

The mean tumor volume of mPEG-SS-20k-GP8 (high modification) group, mPEG-SS-20k-GP8 (moderate modification) group, mPEG-SS-5k-GP8 (high modification) group, mPEG-SS-5k-GP8 (moderate modification) group, mPEG-SS-5k-GP8 (low modification) group at the 17th day after cell inoculation were 550.25±100.23 mm³, 544.90±89.12 mm³, 574.02±108.15 mm³, 676.17±128.23 mm³, 570.45±107.25 mm³, respectively, and both had significant difference compared with model group (P<0.01 or P<0.05). And the tumor growth inhibition value TGI(%) of test sample groups (above samples) was 77%, 80%, 79%, 75%, 79%, respectively.

The mean tumor volume of PEG-SC-20k-rhIL-2 group was 1215.19±184.70 mm³ on the day 17 after cell inoculation. Compared with the model group, there was significant difference in PEG-SC-20k-rhIL-2 group (P<0.05). The tumor growth inhibition value TGI(%) was 57%.

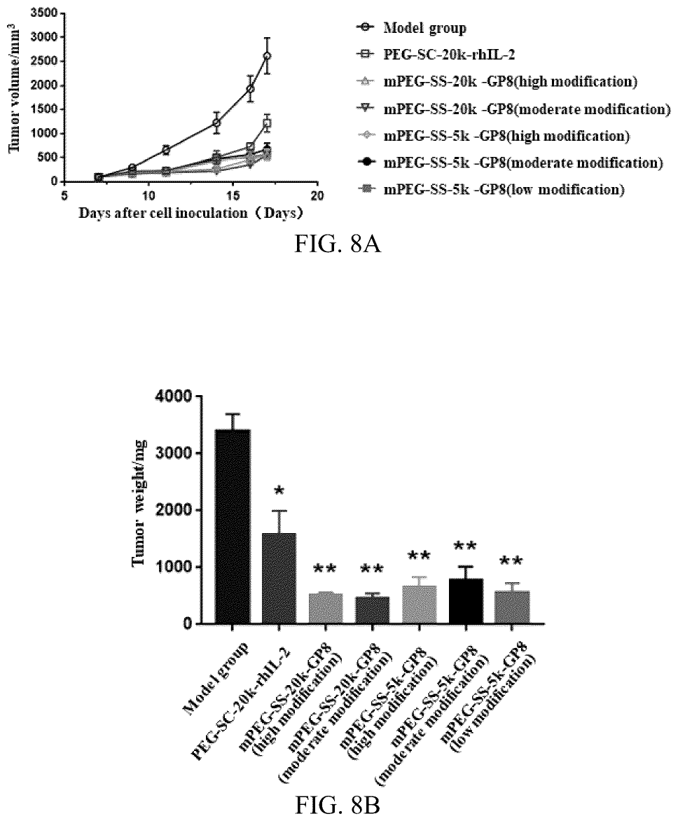

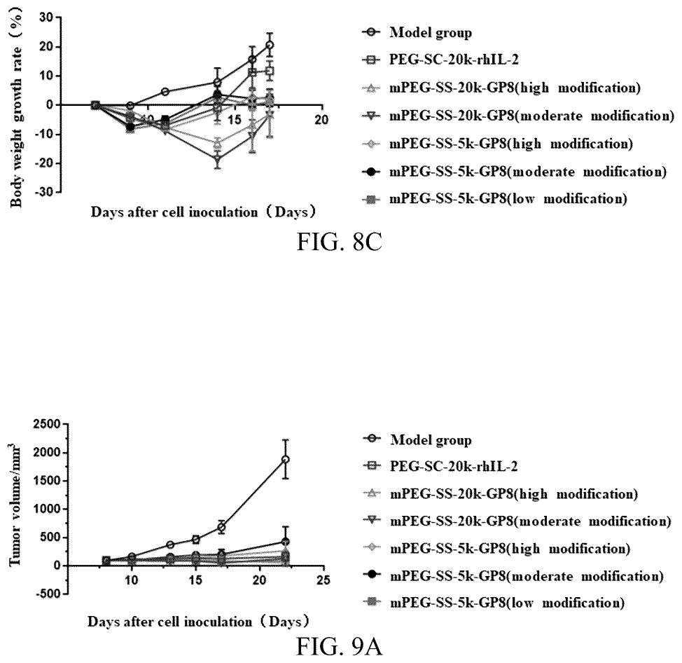

The results of tumor weight were similar to tumor volume. The experimental results were shown in the table below and FIG. 8:

TABLE 18
Tumor volume, relative tumor volume, TGI, and T/C of animals in each group
(Female, Mean ± SEM)

| | | On the day 17 after inoculation(day 3 after the last dose) | | | | |
|---|---|---|---|---|---|---|
| | | | | | | P |
| | | | Relative | | | value(vs |
| | N | Tumor | tumor | TGI | T/C | model |
| Groups | (D17/D1) | volume(mm³) | volume | (%) | (%) | group) |
| Model group | 5/5 | 2,614.97 ± 372.77 | 29.90 ± 4.72 | — | — | — |
| PEG-SC-20k-rhIL-2 | 3/6 | 1,215.19 ± 184.70* | 12.86 ± 3.38 | 57 | 43 | 0.0347 |
| mPEG-SS-20k-GP8 | 2/6 | 550.25 ± 100.23* | 6.75 ± 2.53 | 77 | 23 | 0.0215 |
| (high modification) | | | | | | |
| mPEG-SS-20k-GP8 | 5/6 | 544.90 ± 89.12** | 6.04 ± 1.08** | 80 | 20 | 0.0006 |
| (moderate | | | | | | |
| modification) | | | | | | |
| mPEG-SS-5k-GP8 | 6/6 | 574.02 ± 108.15** | 6.26 ± 0.99** | 79 | 21 | 0.0003 |
| (high modification) | | | | | | |
| mPEG-SS-5k-GP8 | 6/6 | 676.17 ± 128.23** | 7.41 ± 1.22** | 75 | 25 | 0.0005 |
| (moderate | | | | | | |
| modification) | | | | | | |
| mPEG-SS-5k-GP8 | 6/6 | 570.45 ± 107.25** | 6.39 ± 1.03** | 79 | 21 | 0.0003 |
| (low modification) | | | | | | |

Note:
*P < 0.05,
**P < 0.01, vs model group.

TABLE 19
Tumor weight, TGI, T/C of animals in each group (Female, Mean ± SEM)

| | | On the day 17 after cell inoculation(day 3 after the last | | | |
|---|---|---|---|---|---|
| | | dose) | | | |
| | N | | TGI | T/C | P value(vs Model |
| Groups | (D17/D1) | Tumor weight(mg) | (%) | (%) | group) |
| Model group | 5/5 | 3,396.58 ± 300.67 | — | — | — |
| PEG-SC-20k-rhIL-2 | 3/6 | 1,589.87 ± 407.01* | 53 | 47 | 0.0111 |
| mPEG-SS-20k -GP8 | 2/6 | 516.35 ± 52.05 | 85 | 15 | 0.0023 |
| (high modification) | | | | | |
| mPEG-SS-20k -GP8 | 5/6 | 453.60 ± 92.39** | 87 | 13 | 0.0000 |
| (moderate modification) | | | | | |
| mPEG-SS-5k -GP8 | 6/6 | 655.73 ± 181.35** | 81 | 19 | 0.0000 |
| (high modification) | | | | | |
| mPEG-SS-5k -GP8 | 6/6 | 781.92 ± 232.28** | 77 | 23 | 0.0001 |
| (moderate modification) | | | | | |
| mPEG-SS-5k -GP8 | 6/6 | 551.20 ± 172.90** | 84 | 16 | 0.0000 |
| (low modification) | | | | | |

Note:
*P < 0.05, **P < 0.01, vs model group.

### 2. Safety:

In the mPEG-SS-20k-GP8 (high modification) group, one animal showed medium body weight loss (10%<body weight loss≤20%) on the day 4 (on the day 11 after inoculation) after the first dose, two animals showed medium body weight loss (10%<body weight loss≤20%) and 3 animals died on the day 7 (on the day 14 after inoculation) after the first dose. On the day 9 after the first dose (on the day 16 after inoculation), one animal still had medium body weight loss (10%<body weight loss≤20%) and one animal died. At the end of the experiment, the body weight of the two residual animals tended to recover. More than half of the animals in this group died, which was presumed to be related to the test sample.

In mPEG-SS-20k-GP8 (medium modification) group, two animals showed medium body weight loss (10%<body weight loss≤20%) on the day 4 after the first dose (on the day 11 after inoculation). Four animals showed medium body weight loss (10%<body weight loss≤20%) and two animals showed severe weight loss (body weight loss>20%) on the 7th day after the first dose (on the day 14 after inoculation), respectively. On the day 9 after the first dose (on the day 16 after inoculation), one animal showed medium body weight loss (10%<body weight loss≤20%), one animal showed severe body weight loss (body weight loss>20%), and one animal died. Until the end of the experiment, in the residual five animals, four animals showed a tendency to regain weight and one animal did not regain weight. Medium or severe body weight loss and one death were observed in this group, which were presumably related to the test sample.

In the mPEG-SS-5k-GP8 (high modification) group, one animal showed medium body weight loss (10%<body weight loss≤20%) on the day 4 after the first dose (on the day 11 after inoculation). On the day 7 after the first dose (on the day 14 after inoculation) one animal showed medium weight (10%<weight loss≤20%). At the end of the experiment, the body weight of the animals tended to recover.

The body weight of the mPEG-SS-5k-GP8 (moderate modification) group did not change significantly during treatment.

In the mPEG-SS-5k-GP8 (low modification) group, one animal showed medium weight loss (10%<weight loss<20%) on the 4th day after the first dose (on the day 11 after inoculation), and there was recovery trend at the end of the experiment.

In PEG-SC-20k-rhIL-2 group, one animal showed medium body weight loss (10%<weight loss≤20%) on the 4th day after the first dose (on the day 11 after inoculation), and there was a recovery trend at the end of the experiment. 3 animals died on the day 7 after the first dose (on the day 14 after inoculation). Half of the animals in this group died, which was presumed to be related to the test sample.

In the model group, one animal showed medium body weight loss (10%<weight loss≤20%) on the day 7 after the first dose (on the day 14 after inoculation), and had a tendency to recover at the end of the experiment. The specific results were shown in the table below and FIG. 8c.

TABLE 20
Body weight growth rate of animals in each group (Female, Mean ± SEM)

| | | Body weight growth rate(%) | | | | | |
|---|---|---|---|---|---|---|---|
| Groups | N | D7 | D9 | D11 | D14 | D16 | D17 |
| Model group | 5 | 0.00 ± 0.00 | −0.08 ± 0.54 | 4.69 ± 1.02 | 7.92 ± 4.85 | 15.78 ± 4.35 | 20.72 ± 3.98 |
| PEG-SC-20k-rhIL-2 | 3-6 | 0.00 ± 0.00 | −4.20 ± 0.74** | −6.85 ± 0.92** | −0.84 ± 4.41 | 11.29 ± 5.33 | 11.80 ± 3.38 |
| mPEG-SS-20k- | 2-6 | 0.00 ± 0.00 | −1.79 ± 1.03 | −7.56 ± 0.88** | −12.92 ± 1.76* | −6.68 ± 8.96* | −3.03 ± 8.14* |
| GP8(high | | | | | | | |
| modification) | | | | | | | |
| mPEG-SS-20k- | 5-6 | 0.00 ± 0.00 | −3.71 ± 0.92* | −8.75 ± 0.90** | −18.64 ± 3.01** | −10.61 ± 5.67** | −3.43 ± 7.27* |
| GP8(moderate | | | | | | | |
| modification) | | | | | | | |
| mPEG-SS-5k- | 6 | 0.00 ± 0.00 | −4.58 ± 1.19* | −8.34 ± 0.71** | −2.49 ± 3.96 | 2.91 ± 2.68* | 2.05 ± 1.63** |
| GP8(high | | | | | | | |
| modification) | | | | | | | |
| mPEG-SS-5k- | 6 | 0.00 ± 0.00 | −7.21 ± 0.88* | −4.87 ± 1.23** | 3.68 ± 2.85 | 2.39 ± 2.79* | 2.55 ± 3.03** |
| GP8(moderate | | | | | | | |
| modification) | | | | | | | |
| mPEG-SS-5k- | 6 | 0.00 ± 0.00 | −8.14 ± 0.72* | −6.28 ± 1.68** | 2.73 ± 2.23 | 0.12 ± 1.89** | 1.17 ± 1.62* |
| GP8(low | | | | | | | |
| modification) | | | | | | | |

Note:
*P < 0.05,
**P < 0.01, vs model group.

### 3. Analysis of Results

mPEG-SS-20k-GP8 (high modification), mPEG-SS-20k-GP8 (moderate modification), mPEG-SS-5k-GP8 (high modification), mPEG-SS-5k-GP8 (moderate modification) and mPEG-SS-5k-GP8 (low modification) can inhibit the growth of tumor at the dose of 2 mg/kg in B16 F10 mouse melanoma model, which was better than that of V-PEG-SC-20k-rhIL-2.

During the treatment, animals in mPEG-SS-20k-GP8 (high modification) and mPEG-SS-20k-GP8 (moderate modification) groups showed poor tolerance at the dose of 2 mg/kg. Animals in mPEG-SS-5k-GP8 (high modification) group, mPEG-SS-5k-GP8 (low modification) group were basically tolerated at the dose of 2 mg/kg. Animals in mPEG-SS-5k-GP8 (moderate modification) group were well tolerated at the dose of 2 mg/kg.

In this example, it was preliminarily proved that the products with different PEGylation degree had different efficacy and safety in vivo, which was consistent with the results based on in vitro experiments in Example 7. Therefore, the inventors further carried out the comparison work of drug efficacy in other different models.

### Example 14: Pharmacodynamic Evaluation of Linear PEG-SS Modified IL-2 Mutant with Different PEGylation Degree in CT26.WT Murine Colon Cancer Model

### I. Experimental Methods

Experiment method and general clinical observations were the same as example 11. The detailed dosage regimen and administration route were shown in the table below.

TABLE 21
Animal grouping and tumor cell inoculation information

| | | | | The | | |
|---|---|---|---|---|---|---|
| | | | | volume of | | |
| | | | | drug | Administration | Administration |
| Groups | | N | Dose | administered | route | cycle |
| 1 | Model group | 6 | — | — | — | — |
| 2 | PEG-SC-20k-rhIL-2 | 6 | 2 mg/ | 0.1 mL/10 | i.v. | once a week, |
| 3 | mPEG-SS-20k-GP8(high | 6 | kg | g | | for 2 weeks |
| | modification) | | | | | |
| 4 | mPEG-SS-20k-GP8(moderate | 6 | | | | |
| | modification) | | | | | |
| 5 | mPEG-SS-5k-GP8(high | 6 | | | | |
| | modification) | | | | | |
| 6 | mPEG-SS-5k-GP8(moderate | 6 | | | | |
| | modification) | | | | | |
| 7 | mPEG-SS-5k-GP8(low | 6 | | | | |
| | modification) | | | | | |

Note:
N: number of animals;
s.c.: subcutaneous injection;
i.v.: intravenous injection.
The day of cell inoculation was D1.

### II. Experimental Results

### 1, Efficacy:

The mean tumor volume of the model group was 1,886.67±341.33 mm³ on the day 22 after cell inoculation.

The mean tumor volume of mPEG-SS-20k-GP8 (high modification) group, mPEG-SS-20k-GP8 (moderate modification) group, mPEG-SS-5k-GP8 (high modification) group, mPEG-SS-5k-GP8 (moderate modification) group, mPEG-SS-5k-GP8 (low modification) group at the 22nd day after cell inoculation were 268.15±163.54 mm³, 132.07±132.07 mm³, 56.90±36.21 mm³, 429.12±261.88 mm³, 87.78±87.78 mm³, respectively. There was significant difference compared with model group (P<0.01). The tumor growth inhibition value of test sample groups (above samples) (TGI %) were 88%, 90%, 97%, 80% and 95%, respectively.

The mean tumor volume of PEG-SC-20k-rhIL-2 group at the 22nd day after cell inoculation was 163.13±73.03 mm³, which was significantly different (P<0.01) with model group. The tumor growth inhibition value TGI (%) was 92%.

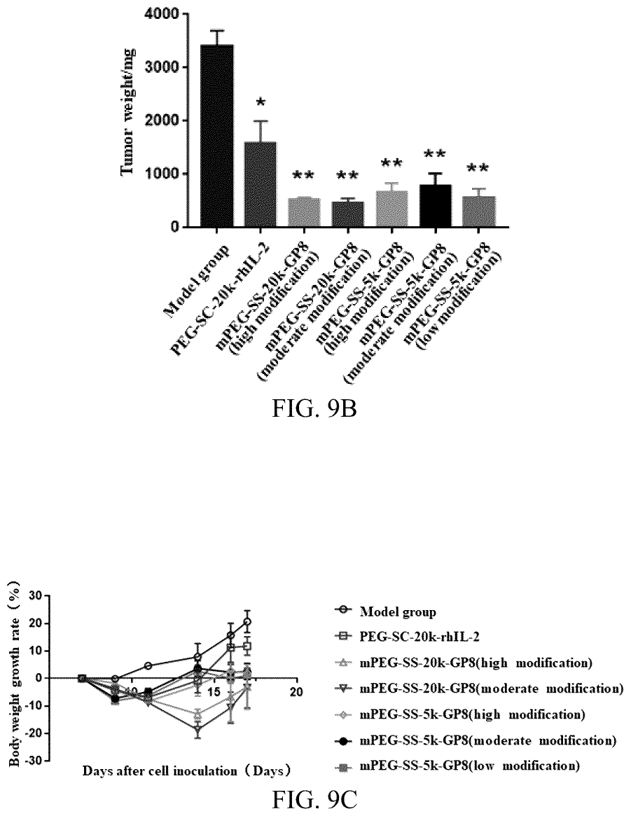

The results of tumor weight were similar to those of tumor volume. The experimental results were shown in the table below and FIG. 9:

TABLE 22
Tumor volume, relative tumor volume, TGI, T/C of animals in each group (Female,
Mean ± SEM)

| | | On the day 22 after inoculation(day 7 after the last dose) | | | | |
|---|---|---|---|---|---|---|
| | N | | Relative tumor | TGI | | P value(vs |
| Groups | (D22/D1) | Tumor volume(mm³) | volume | (%) | T/C (%) | model group) |
| Model group | 6/6 | 1,886.67 ± 341.33 | 18.95 ± 3.02 | — | — | — |
| PEG-SC-20k-rhIL-2 | 6/6 | 163.13 ± 73.03 ** | 1.58 ± 0.74 | 92 | 8 | 0.0006 |
| mPEG-SS-20k-GP8 | 5/6 | 268.15 ± 163.54 ** | 2.26 ± 1.17 | 88 | 12 | 0.0031 |
| (high modification) | | | | | | |
| mPEG-SS-20k-GP8 | 5/6 | 132.07 ± 132.07 ** | 1.83 ± 1.83 | 90 | 10 | 0.0016 |
| (moderate modification) | | | | | | |
| mPEG-SS-5k-GP8 | 6/6 | 56.90 ± 36.21 ** | 0.66 ± 0.44 | 97 | 3 | 0.0003 |
| (high modification) | | | | | | |
| mPEG-SS-5k-GP8 | 6/6 | 429.12 ± 261.88 ** | 3.82 ± 1.96 | 80 | 20 | 0.0069 |
| (moderate modification) | | | | | | |
| mPEG-SS-5k-GP8 | 6/6 | 87.78 ± 87.78 ** | 0.96 ± 0.96 | 95 | 5 | 0.0005 |
| (low modification) | | | | | | |

Note:
* P < 0.05,
** P < 0.01, vs model group.

TABLE 23
Tumor weight, TGI, and T/C of animals in each group (Female, Mean ± SEM)

| | | On the day 22 after cell inoculation(day 7 after the last dose) | | | |
|---|---|---|---|---|---|
| | N | | TGI | | P value(vs |
| Groups | (D22/D1) | Tumor weight(mg) | (%) | T/C (%) | model group) |
| Model group | 6/6 | 1,619.53 ± 292.62 | — | — | — |
| PEG-SC-20k-rhIL-2 | 6/6 | 105.57 ± 48.04** | 93 | 7 | 0.0005 |
| mPEG-SS-20k -GP8 | 5/6 | 180.36 ± 116.55** | 89 | 11 | 0.0022 |
| (high modification) | | | | | |
| mPEG-SS-20k -GP8 | 5/6 | 97.36 ± 97.36** | 94 | 6 | 0.0014 |
| (moderate | | | | | |
| modification) | | | | | |
| mPEG-SS-5k -GP8 | 6/6 | 34.52 ± 21.86** | 98 | 2 | 0.0003 |
| (high modification) | | | | | |
| mPEG-SS-5k - | 6/6 | 303.72 ± 179.03** | 81 | 19 | 0.0033 |
| GP8(moderate | | | | | |
| modification) | | | | | |
| mPEG-SS-5k -GP8 | 6/6 | 82.95 ± 82.95** | 95 | 5 | 0.0005 |
| (low modification) | | | | | |

Note:
*P < 0.05, **P < 0.01, vs model group.

### 2. Safety:

In the mPEG-SS-20k-GP8 (high modification) group, three animals showed moderate body weight loss (10%<weight loss≤20%) on the day 5 after the first dose, and one animal died. Moderate body weight loss (10%<weight loss≤20%) occurred in two animals on the day 2 after the second dose and tended to recover by the end of the experiment.

mPEG-SS-20k-GP8 (moderate modification) group showed moderate body weight loss (10%<weight loss≤20%) in three animals on day 5 after the first dose, one animal died. Moderate body weight loss (10%<weight loss≤20%) occurred in one animal on day 7 after the second dose. Moderate body weight loss (10%<weight loss≤20%) occurred in one animal on the day 2 after the second dose and did not recover by the end of the experiment.

mPEG-SS-5k-GP8 (high modification) group showed moderate body weight loss (10%<weight loss≤20%) in one animal on the day 5 after the first dose. Moderate body weight loss (10%<weight loss≤20%) occurred in one animal on the day 2 after the second dose and tended to recover by the end of the experiment.

mPEG-SS-5k-GP8 (moderate modification) showed no weight loss or less than 10% weight loss during the treatment, and the weight loss recovered later in the experiment.

mPEG-SS-5k-GP8 (low modification) showed moderate body weight loss (10%<weight loss≤20%) in one animal on the day 2 after the second dose, which tended to be recovered by the end of the experiment.

In PEG-SC-20k-rhIL-2 group, four animals showed moderate body weight loss (10%<weight loss≤20%) on the day 5 after the first dose. Moderate body weight loss (10%<weight loss≤20%) was observed in two animals on the day 2 after the second dose. Moderate body weight loss (10%<weight loss≤20%) was still observed in one animal on the day 7 after the second dose. The rest of the animals had a tendency to recover until the end of the experiment.

TABLE 24
Body weight growth rate of animals in each group (Female, Mean ± SEM)

| | | Body weight growth rate (%) | | | | | |
|---|---|---|---|---|---|---|---|
| Groups | N | D8 | D10 | D13 | D15 | D17 | D22 |
| Model group | 6 | 0.00 ± 0.00 | −0.94 ± 0.69 | −0.18 ± 0.32 | 5.04 ± 0.89 | 4.54 ± 0.45 | 7.71 ± 0.85 |
| PEG-SC-20k- | 6 | 0.00 ± 0.00 | −2.64 ± 0.51 | −11.67 ± 1.86** | −1.45 ± 2.66* | −8.01 ± 2.32** | −1.92 ± 3.40* |
| rhIL-2 | | | | | | | |
| mPEG-SS-20k- | 5-6 | 0.00 ± 0.00 | 0.43 ± 0.87 | −9.30 ± 3.27* | −1.07 ± 2.81 | −6.53 ± 2.15** | 1.61 ± 3.45 |
| GP8(high | | | | | | | |
| modification) | | | | | | | |
| mPEG-SS-20k- | 5-6 | 0.00 ± 0.00 | −1.60 ± 0.88 | −10.71 ± 2.45** | −2.50 ± 3.03* | −7.42 ± 3.12** | −0.49 ± 4.32 |
| GP8(moderate | | | | | | | |
| modification) | | | | | | | |
| mPEG-SS-5k- | 6 | 0.00 ± 0.00 | −3.93 ± 0.69* | −5.32 ± 1.70* | 1.80 ± 2.11 | −1.94 ± 2.11* | 0.51 ± 1.91* |
| GP8(high | | | | | | | |
| modification) | | | | | | | |
| mPEG-SS-5k- | 6 | 0.00 ± 0.00 | −2.99 ± 0.79 | −1.80 ± 1.52 | 3.61 ± 1.02 | −0.52 ± 2.35 | 2.88 ± 2.16 |
| GP8(moderate | | | | | | | |
| modification) | | | | | | | |
| mPEG-SS-5k- | 6 | 0.00 ± 0.00 | −7.31 ± 0.51** | −4.55 ± 1.11** | −0.02 ± 1.25** | −6.20 ± 1.36** | −0.42 ± 1.17** |
| GP8(low | | | | | | | |
| modification) | | | | | | | |

Note:
*P < 0.05,
**P < 0.01, vs model group.

### 3. Analysis of Results

mPEG-SS-20k-GP8 (high modification), mPEG-SS-20k-GP8 (moderate modification), mPEG-SS-5k-GP8 (high modification), mPEG-SS-5k-GP8 (moderate modification), mPEG-SS-5k-GP8 (low modification) can inhibit the tumor growth in CT26.WT murine colon cancer model at the dose of 2 mg/kg.

During the treatment, animals in mPEG-SS-5k-GP8 (high modification), mPEG-SS-5k-GP8 (moderate modification) and mPEG-SS-5k-GP8 (low modification) group were well tolerated at the dose of 2 mg/kg. Animals in the mPEG-SS-20k-GP8 (high modification) and mPEG-SS-20k-GP8 (moderate modification) groups were basically tolerated at the dose of 2 mg/kg, and body weight recovery was better than that of V-PEG-SC-20k-rhIL-2.

### Example 15: Pharmacodynamic Evaluation of Linear PEG-SS Modified IL-2 Mutant with Different PEGylation Degree in A375 Human Melanoma Tumor Model

### I. Experimental Methods

A375 cells were cultured in DMEM medium containing 10% fetal bovine serum. A375 cells in exponential growth phase were collected and resuspended in PBS. hu-PBMCs (human peripheral blood mononuclear cells) were cultured in 1640 medium containing 10% fetal bovine serum.

PBMCs were collected at the third day after stimulated with OKT-3 and IL-2, and resuspended in PBS. 5×10⁵ cells/0.1 ml A375 cells and 5×10⁵ cells/0.1 ml human PBMCs were mixed in equal volume, and were inoculated subcutaneously into the right flank of NOD/SCID mice, 0.2 mL per mouse. On the day after cell inoculation, the animals were divided into groups according to the body weight, and the first dose was started on the day of grouping. The detailed dosage regimen and administration route were shown in the table below.

TABLE 25
The animal grouping and tumor cell inoculation information

| | | | | The | | |
|---|---|---|---|---|---|---|
| | | | | volume of | | |
| | | | | drug | Administration | Administration |
| Groups | | N | Dose | administered | route | cycle |
| 1 | Model group | 5 | — | — | — | — |
| 2 | PEG-SC-20k-rhIL-2 | 5 | 0.5 mg/ | 0.1 mL/10 | i.v. | once a |
| 3 | mPEG-SS-20k-GP8(high | 6 | kg | g | | week, for |
| | modification) | | | | | 5 weeks |
| 4 | mPEG-SS-20k-GP8(moderate | 6 | | | | |
| | modification) | | | | | |
| 5 | mPEG-SS-5k-GP8(high | 6 | | | | |
| | modification) | | | | | |
| 6 | mPEG-SS-5k-GP8(moderate | 6 | | | | |
| | modification) | | | | | |
| 7 | mPEG-SS-5k-GP8(low | 6 | | | | |
| | modification) | | | | | |

Note:
N: number of animals;
s.c.: subcutaneous injection;
i.v.: intravenous injection.
The day of cell inoculation was D1.

The detection of general clinical observations, body weight, tumor volume, tumor weight were the same with example 11.

### II. Experimental Results

### 1, Efficacy:

The mean tumor volume of the model group was 1,970.97±298.65 mm³ on the day 45 after cell inoculation.

The mPEG-SS-20k-GP8 (high modification) group, mPEG-SS-20k-GP8 (moderate modification) group and mPEG-SS-5k-GP8 (moderate modification) group showed no tumor growth on the day 45 after cell inoculation, and both had significant difference compared with model group (P<0.01). The mean tumor volume of PEG-SC-20k-rhIL-2, mPEG-SS-5k-GP8 (high modification), mPEG-SS-5k-GP8 (low modification) group was 136.59±134.84 mm³, 83.34±83.34 mm³, 96.62±64.92 mm³, having significant difference (P<0.01) with the model group respectively.

Results of tumor weight were close to the results of tumor volume. And TGI (based on tumor weight) of mPEG-SS-20k-GP8 (high modification) group, mPEG-SS-20k-GP8 (moderate modification) group, mPEG-SS-5k-GP8 (high modification) group, mPEG-SS-5k-GP8 (moderate modification) group, mPEG-SS-5k-GP8 (low modification) group, PEG-SC-20k-rhIL-2 group were 100%, 100%, 95%, 100%, 95% and 91%, respectively.

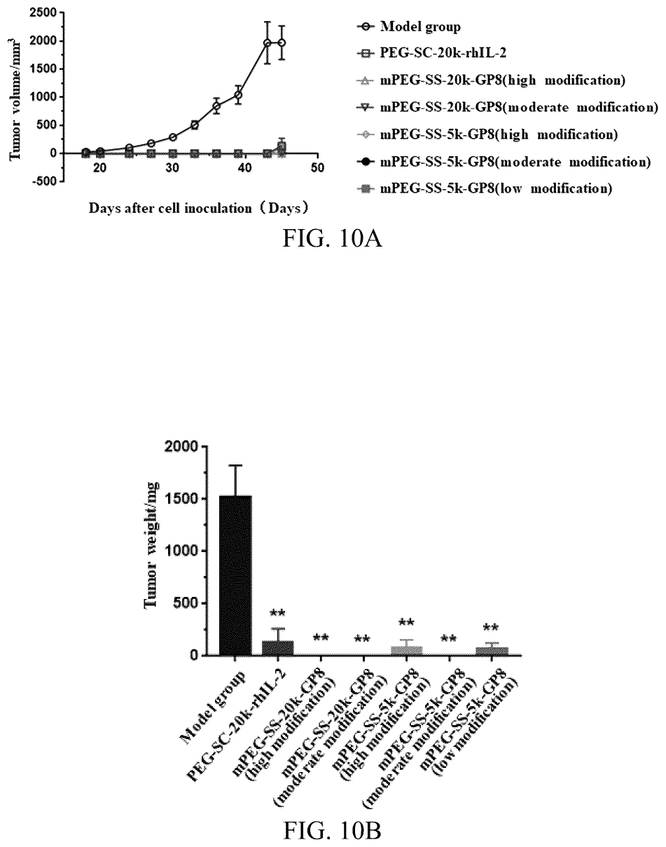

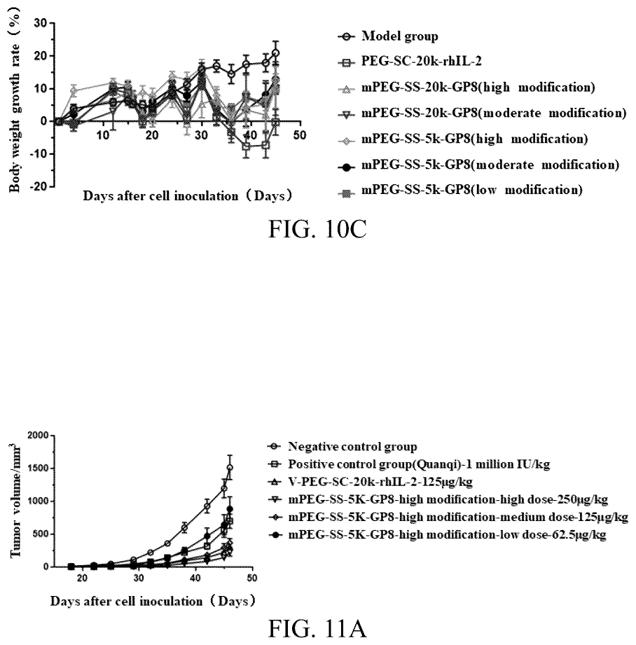

The specific experimental results were shown in the following table and FIG. 10:

TABLE 26
Tumor volume, TGI, and T/C of animals in each group at day 45 after cell inoculation
(Female, Mean ± SEM)

| | | on the day 45 after cell inoculation | | | |
|---|---|---|---|---|---|
| | | | | | P value(vs Model |
| Groups | n | tumor volume(mm³) | T/C | TGI(%) | group) |
| Model group | 5/5 | 1,970.97 ± 298.65 | — | — | — |
| PEG-SC-20k-rhIL-2 | 4/5 | 136.59 ± 134.84** | 7 | 93 | 0.001 |
| mPEG-SS-20k-GP8(high | 5/6 | 0.00 ± 0.00** | 0 | 100 | 0.000 |
| modification) | | | | | |
| mPEG-SS-20k-GP8(moderate | 4/6 | 0.00 ± 0.00** | 0 | 100 | 0.001 |
| modification) | | | | | |
| mPEG-SS-5k-GP8(high | 6/6 | 83.34 ± 83.34** | 4 | 96 | 0.000 |
| modification) | | | | | |
| mPEG-SS-5k-GP8(moderate | 6/6 | 0.00 ± 0.00** | 0 | 100 | 0.000 |
| modification) | | | | | |
| mPEG-SS-5k-GP8(low | 6/6 | 96.62 ± 64.92** | 5 | 95 | 0.000 |
| modification) | | | | | |

Note:
**P < 0.01, vs model group; *P < 0.05, vs model group.

TABLE 27
Tumor weight of animals in each group on day 45 after cell inoculation
(Female, Mean ± SEM)

| | | on the day 45 after cell inoculation | | | |
|---|---|---|---|---|---|
| | | | | | P value |
| | | Tumor Weight | | | (vs Model |
| Groups | n | (mg) | T/C | TGI(%) | group) |
| Model group | 5/5 | 1,520.68 ± 304.64 | — | — | — |
| PEG-SC-20k-rhIL-2 | 4/5 | 130.68 ± 130.68** | 9 | 91 | 0.007 |
| mPEG-SS-20k-GP8(high | 5/6 | 0.00 ± 0.00** | 0 | 100 | 0.001 |
| modification) | | | | | |
| mPEG-SS-20k-GP8(moderate | 4/6 | 0.00 ± 0.00** | 0 | 100 | 0.003 |
| modification) | | | | | |
| mPEG-SS-5k-GP8(high | 6/6 | 77.93 ± 77.93** | 5 | 95 | 0.001 |
| modification) | | | | | |
| mPEG-SS-5k-GP8(moderate | 6/6 | 0.00 ± 0.00** | 0 | 100 | 0.000 |
| modification) | | | | | |
| mPEG-SS-5k-GP8(low | 6/6 | 74.60 ± 48.58** | 5 | 95 | 0.001 |
| modification) | | | | | |

Note:
**P < 0.01, vs model group; *P < 0.05, vs model group.

TABLE 28
Body weight growth rate of animals in each group (Female, Mean ± SEM)

| | | Body weight growth rate(%) | | | | | | | |
|---|---|---|---|---|---|---|---|---|---|
| Groups | n | D1 | D4 | D12 | D15 | D16 | D18 | D20 | D24 |
| Model group | 5 | 0.00 ± | 4.02 ± | 6.01 ± | 6.45 ± | 5.41 ± | 5.28 ± | 4.42 ± | 8.89 ± |
| | | 0.00 | 1.32 | 1.04 | 0.61 | 0.98 | 1.16 | 1.34 | 1.86 |
| PEG-SC-20k-rhIL-2 | 4-5 | 0.00 ± | −0.77 ± | 9.97 ± | 8.79 ± | 5.62 ± | 0.08 ± | 3.24 ± | 8.46 ± |
| | | 0.00 | 1.19* | 1.72 | 1.15 | 1.17 | 1.65* | 1.54 | 1.15 |
| mPEG-SS-20k-GP8 | 5-6 | 0.00 ± | 4.08 ± | 6.69 ± | 9.14 ± | 6.85 ± | 2.38 ± | 0.47 ± | 7.06 ± |
| (high modification) | | 0.00 | 1.36 | 1.97 | 2.07 | 1.76 | 1.73 | 2.09 | 2.69 |
| mPEG-SS-20k-GP8 | 4-6 | 0.00 ± | −1.31 ± | 3.08 ± | 8.17 ± | 6.62 ± | 0.60 ± | 2.76 ± | 11.28 ± |
| (moderate modification) | | 0.00 | 1.64* | 5.68 | 3.05 | 1.40 | 2.46 | 2.42 | 1.08 |
| mPEG-SS-5k-GP8 | 6 | 0.00 ± | 9.52 ± | 12.01 ± | 10.96 ± | 7.69 ± | 8.88 ± | 8.04 ± | 13.90 ± |
| (high modification) | | 0.00 | 1.85* | 1.31** | 1.83 | 2.01 | 2.21 | 2.30 | 1.57 |
| mPEG-SS-5k-GP8 | 6 | 0.00 ± | 2.30 ± | 10.10 ± | 10.64 ± | 7.62 ± | 3.21 ± | 6.15 ± | 10.37 ± |
| (moderate modification) | | 0.00 | 2.28 | 1.43 | 1.55* | 1.87 | 1.15 | 1.41 | 2.34 |
| mPEG-SS-5k-GP8 (low | 6 | 0.00 ± | −0.21 ± | 9.30 ± | 7.13 ± | 5.24 ± | 0.96 ± | 3.49 ± | 8.39 ± |
| modification) | | 0.00 | 2.63 | 1.08 | 0.42 | 0.69 | 0.75* | 0.86 | 0.52 |

Note:
** P < 0.01, vs model group;
* P < 0.05, vs model group.

TABLE 29
Body weight growth rate of animals in each group (Female, Mean ± SEM)

| | | Body weight growth rate(%) | | | | | | |
|---|---|---|---|---|---|---|---|---|
| Groups | n | D27 | D30 | D33 | D36 | D39 | D43 | D45 |
| Model group | 5 | 11.41 ± | 15.96 ± | 17.05 ± | 14.59 ± | 17.58 ± | 18.04 ± | 21.08 ± |
| | | 1.69 | 1.99 | 1.83 | 2.93 | 2.73 | 2.78 | 3.54 |
| PEG-SC-20k-rhIL-2 | 4- | 1.26 ± | 12.17 ± | 1.47 ± | −3.19 ± | −7.56 ± | −7.17 ± | −0.18 ± |
| | 5 | 1.72** | 2.22 | 2.70** | 3.21** | 3.52** | 3.88** | 3.92** |
| mPEG-SS-20k-GP8(high | 5- | −0.66 ± | 5.47 ± | 7.09 ± | 0.17 ± | 3.61 ± | 2.01 ± | 10.52 ± |
| modification) | 6 | 3.37* | 4.31 | 2.80* | 2.58** | 5.57 | 5.73* | 6.31 |
| mPEG-SS-20k-GP8 | 4- | 1.86 ± | 12.47 ± | 2.86 ± | −0.26 ± | 7.50 ± | 4.90 ± | 10.15 ± |
| (moderate modification) | 6 | 2.57* | 2.38 | 3.78** | 4.90* | 6.87 | 7.71 | 8.21 |
| mPEG-SS-5k-GP8(high | 6 | 12.99 ± | 16.83 ± | 8.16 ± | 2.50 ± | 4.43 ± | 5.38 ± | 12.76 ± |
| modification) | | 2.23 | 2.30 | 2.65* | 3.19* | 3.43* | 2.00** | 1.91 |
| mPEG-SS-5k-GP8 | 6 | 8.12 ± | 12.30 ± | 3.79 ± | −0.27 ± | 3.65 ± | 8.24 ± | 12.95 ± |
| (moderate modification) | | 1.88 | 2.20 | 1.85** | 2.97** | 5.05* | 3.62 | 4.45 |
| mPEG-SS-5k-GP8(low | 6 | 5.44 ± | 11.26 ± | 4.77 ± | 3.93 ± | 7.74 ± | 5.83 ± | 10.23 ± |
| modification) | | 0.88** | 0.73* | 0.71** | 1.48** | 1.59** | 1.18** | 1.11* |

Note:
**P < 0.01, vs model group;
*P < 0.05, vs model group.

### 2. Safety:

mPEG-SS-20k-GP8 (high modification) showed moderate body weight loss (10%<body weight loss≤20%) in one animal on the day 3 after the fourth dose, this animal maintained moderate weight loss (10%<body weight loss≤20%) on the day 6 after the fourth dose, and died on the day 1 after the last dose. Moderate body weight loss (10%<body weight loss≤20%) was observed in one animal from the 9th day to the 13th day after the last dose, and the body weight of the animals tended to recover at the end of the experiment.

mPEG-SS-20k-GP8 (moderate modification) group showed severe body weight loss (body weight loss>20%) in one animal at the 3rd day after the second dose and died at the 6th day after the second dose. Moderate body weight loss (10%<body weight loss≤20%) was observed in one animal from the third day after the last dose to the end of the experiment. At the 4th day after the last dose, one animal died.

mPEG-SS-5k-GP8 (moderate modification) showed moderate body weight loss (10%<body weight loss≤20%) in one animal at the 6th day to the 9th day after the last dose, and the body weight of the animals tended to regain by the end of the experiment.

mPEG-SS-5k-GP8 (high modification) and mPEG-SS-5k-GP8 (low modification) showed no weight loss or less than 10% weight loss during the treatment, and the body weight loss of animals recovered later in the experiment.

In PEG-SC-20k-rhIL-2 group, one animal showed moderate body weight loss (10%<weight loss≤20%) at the 6th day after the last dose. Moderate body weight loss (10%<weight loss≤20%) was observed in 2 animals at the 9th day after the last dose. At the 13th day after the last dose two animals showed moderate weight loss (10%<weight loss≤20%), one animal died.

### 3. Analysis of Results

Test samples mPEG-SS-20k-GP8 (high modification), mPEG-SS-20k-GP8 (moderate modification), mPEG-SS-5k-GP8 (high modification), mPEG-SS-5k-GP8 (moderate modification), mPEG-SS-5k-GP8 (low modification) can inhibit the growth of tumor at the dose of 0.5 mg/kg (once a week, for 5 weeks) in A375 human melanoma model. In terms of efficacy, the test samples mPEG-SS-20k-GP8 (high modification), mPEG-SS-20k-GP8 (moderate modification), mPEG-SS-5k-GP8 (high modification), mPEG-SS-5k-GP8 (moderate modification), mPEG-SS-5k-GP8 (low modification) showed slightly better effects than PEG-SC-20k-rhIL-2.

During the treatment, animals in mPEG-SS-20k-GP8 (high modification), mPEG-SS-20k-GP8 (moderate modification) and V-PEG-SC-20k-rhIL-2 group were poorly tolerated at the dose of 0.5 mg/kg. Animals in mPEG-SS-5k-GP8 (high modification) and mPEG-SS-5k-GP8 (low modification) group were well tolerated at the dose of 0.5 mg/kg (once a week, for 5 weeks). Animals in mPEG-SS-5k-GP8 (moderate modification) group were basically tolerated at the dose of 0.5 mg/kg (once a week, for 5 weeks). In terms of safety (body weight and death), mPEG-SS-5k-GP8 (high modification), mPEG-SS-5k-GP8 (moderate modification) and mPEG-SS-5k-GP8 (low modification) were better than PEG-SC-20k-rhIL-2.

### Example 16: Pharmacodynamic Evaluation of PEG Modified IL-2 Mutants in A375 Human Melanoma Model

### I. Experimental Methods

A375 cells were cultured in DMEM medium containing 10% fetal bovine serum. A375 cells in exponential growth phase were collected and resuspended in PBS. hu-PBMCs (human peripheral blood mononuclear cells) were cultured in 1640 medium containing 10% fetal bovine serum. PBMCs were collected on the third day after stimulated with OKT-3 and IL-2 and resuspended in PBS. 1×10⁶ cells/0.1 ml A375 cells and 1×10⁶ cells/0.1 ml human PBMCs were mixed in equal volume, and were inoculated subcutaneously into the right flank of NOD/SCID mice, 0.2 mL per mouse. On the day after cell inoculation, the animals were divided into groups according to body weight, and the first dose was started on the day of grouping; the detailed dosage regimen and administration route were shown in the table below.

TABLE 30
Animal grouping and tumor cell inoculation information

| | | | | The | | |
|---|---|---|---|---|---|---|
| | | | | volume of | | |
| | | | | drug | Administration | Administration |
| Groups | | N | Dose | administered | route | cycle |
| 1 | Negative control group | 8 | Normal | — | — | — |
| | | | saline | | | |
| | | | for | | | |
| | | | injection | | | |
| 2 | Positive control group(Quanqi)- | 8 | 1 | 10 mL/kg | i.v. | Once a day, |
| | 1 million IU/kg | | million | | | for 20 days |
| | | | IU/kg | | | |
| 3 | V-PEG-SC-20k-rhIL-2- | 8 | 125 μg/ | | | Once a |
| | 125 μg/kg | | kg | | | week, for 5 |
| 4 | mPEG-SS-5K-GP8-high | 8 | 250 μg/ | | | weeks |
| | modification | | kg | | | |
| | high dose-250 μg/kg | | | | | |
| 5 | mPEG-SS-5K-GP8-high | 8 | 125 μg/ | | | |
| | modification | | kg | | | |
| | medium dose-125 μg/kg | | | | | |
| 6 | mPEG-SS-5K-GP8-high | 8 | 62.5 μg/ | | | |
| | modification | | kg | | | |
| | low dose-62.5 μg/kg | | | | | |

Note:
N: number of animals;
i.v.: intravenous administration.
The day of cell inoculation was D1. In terms of IL-2 activity units, the total dose of positive control group (Quanqi, recombinant human interleukin-2 for injection (125Ser)) was 1.4 times that of mPEG-SS-5K-GP8-high modification (high dose) group.

The detection of general clinical observations, body weight, tumor volume, and tumor weight were the same as in Example 11.

### II. Experimental Results

1, Efficacy:

The mean tumor volume of negative control group was 1197.64±143.79 mm³ at the 45th day after inoculation.

The mean tumor volume of mPEG-SS-5K-GP8-high modification (250 μg/kg, 125 μg/kg, 62.5 g/kg) were 144.87±35.11 mm³, 296.04±61.10 mm³, 043.89±153.05 mm³ at the 45th day after cell inoculation, respectively. Compared with the negative control group, the differences of mPEG-SS-5K-GP8-high modification (250 μg/kg, 125 μg/kg, 62.5 g/kg) were significant respectively (P<0.01 or P<0.05).

The mean tumor volume of positive control group (Quanqi, 1 million IU/kg) was 557.54±104.82 mm³ at the 45th day after cell inoculation, being significantly different from negative control group (P<0.01). Compared with the positive control group (Quanqi), the tumor volume of mPEG-SS-5K-GP8-high modification (high dose, 250 μg/kg) group was significantly different (P<0.01). The results showed that the total dose of positive control group (Quanqi) was higher than that of mPEG-SS-5K-GP8-high modification (high dose, 250 μg/kg) group, but mPEG-SS-5K-GP8-high modification (high dose, 250 μg/kg) had better drug effect.

The mean tumor volume of positive reference drug (V-PEG-SC-20k-rhIL-2, imitation NKTR 214) was 224.37±33.28 mm³ at the 45th day after cell inoculation, being significantly different from negative control group (P<0.01). There was no significant difference in tumor volume between mPEG-SS-5K-GP8-high modification (medium dose) group and positive reference group (P>0.05). The results showed that the efficacy of mPEG-SS-5K-GP8-high modification was similar to that of positive reference drug (V-PEG-SC-20k-rhIL-2, imitation NKTR214) with the same dose.

Tumor weight results were similar to tumor volume. The TGI of mPEG-SS-5k-GP8-high modification group (high, medium and low dose) based on tumor weight were 87%, 75%, 46% respectively. The TGI of positive control group (Quanqi) and positive reference group (V-PEG-SC-20k-rhIL-2, imitation NKTR214) were 56% and 81%, respectively.

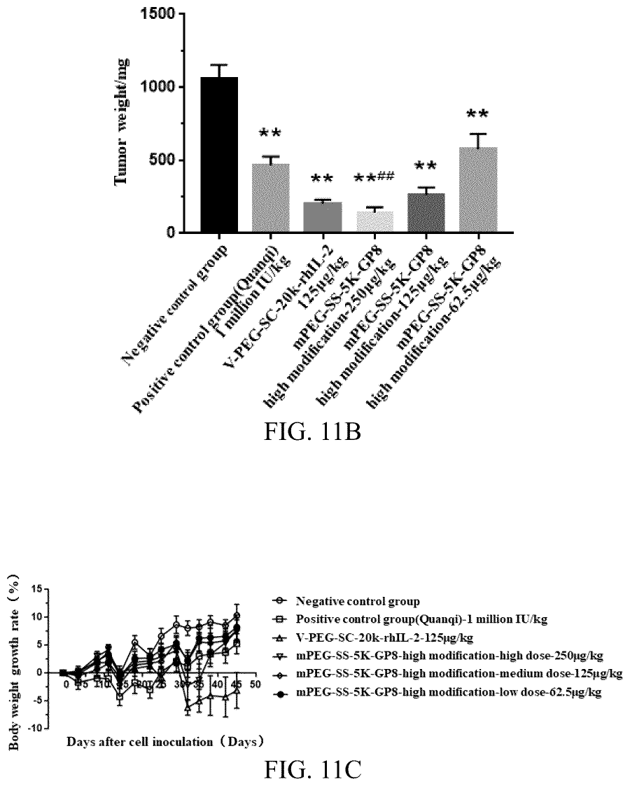

The specific experimental results were shown in the following table and FIG. 11:

TABLE 31
Tumor volume of animals in each group at the 45th day after
cell inoculation (Female, Mean ± SEM)

| | | At the 45th day after cell inoculation | |
|---|---|---|---|
| | | | P value |
| | | | (vs |
| Groups | n | Tumor volume(mm³) | Model) |
| Negative control group | 8 | 1,197.64 ± 143.79 | — |
| Positive control | 8 | 557.54 ± 104.82** | 0.003 |
| group(Quanqi)- | | | |
| 1 million IU/kg | | | |
| V-PEG-SC-20k-rhIL-2- | 8 | 224.37 ± 33.28** | 0.000 |
| 125 μg/kg | | | |
| mPEG-SS-5K -GP8- | 8 | 144.87 ± 35.11**## | 0.000 |
| high modification | | | |
| high dose -250 μg/kg | | | |
| mPEG-SS-5K -GP8- | 8 | 296.04 ± 61.10** | 0.000 |
| high modification | | | |
| medium dose -125 μg/kg | | | |
| mPEG-SS-5K -GP8- | 8 | 643.88 ± 153.05* | 0.020 |
| high modification | | | |
| low dose-62.5 μg/kg | | | |

Note:
**P < 0.01, vs negative control; *P < 0.05, vs the negative control ##P < 0.01, vs positive control (Quanqi).
In terms of IL-2 activity units, the total dose of positive control group (Quanqi, recombinant human interleukin-2 for injection (125Ser)) was 1.4 times that of mPEG-SS-5K-GP8-high modification (high dose) group.

TABLE 32
Tumor weight of animals in each group at the 45th day after cell inoculation
(Female, Mean ± SEM)

| | | At the 45th day after cell inoculation | | | |
|---|---|---|---|---|---|
| | | | T/C | | P value(vs |
| Groups | n | Tumor weight(mg) | (%) | TGI(%) | Model) |
| Negative control group | 8 | 1,055.91 ± 99.00 | — | — | — |
| Positive control group(Quanqi)- | 8 | 465.25 ± 59.86** | 44 | 56 | 0.000 |
| 1 million IU/kg | | | | | |
| V-PEG-SC-20k-rhIL-2-125 μg/kg | 8 | 201.96 ± 28.06** | 19 | 81 | 0.000 |
| mPEG-SS-5K -GP8- high | 8 | 142.24 ± 36.68**## | 13 | 87 | 0.000 |
| modification high dose- | | | | | |
| 250 μg/kg | | | | | |
| mPEG-SS-5K -GP8- high | 8 | 261.00 ± 52.72** | 25 | 75 | 0.000 |
| modification medium dose- | | | | | |
| 125 μg/kg | | | | | |
| mPEG-SS-5K -GP8- high | 8 | 573.86 ± 108.41** | 54 | 46 | 0.005 |
| modification low dose-62.5 μg/kg | | | | | |

Note:
**P < 0.01, vs the negative control; *P < 0.05, vs negative control.
##P < 0.01, vs positive control (Quanqi).
In terms of IL-2 activity units, the total dose of positive control group (Quanqi, recombinant human interleukin-2 for injection (125Ser)) was 1.4 times that of mPEG-SS-5K-GP8-high modification (high dose) group.

### 2. Safety:

In the mPEG-SS-5K-GP8-high modification (high dose) group (250 μg/kg), 3 animals showed moderate weight loss during the treatment, and the animals were generally tolerant to the treatment. mPEG-SS-5k-GP8-high modification groups at the dose of 125 μg/kg, 62.5 μg/kg did not show obvious drug toxicity during the treatment, and the animals were well tolerated during the treatment.

Positive control group (Quanqi, 1 million IU/kg) did not show obvious drug toxicity, animals were well tolerated during the treatment. At the 3rd day (D332) and the 16th day (D45) after the last dose, the mPEG-SS-5K-GP8-high modification (high dose) group (250 μg/kg) had no statistical difference (P>0.05) in body weight with the positive control group (Quanqi, 1 million I/kg).

In V-PEG-SC-20k-rhIL-2 group (125 μg/kg), 3 animals had moderate weight loss, 1 animal had moderate to severe weight loss, and the animals were generally tolerant during the treatment. At the 3rd day (D332) and the 16th day (D45) after the last dose, mPEG-SS-5k-GP8-high modification (medium dose) group (125 μg/kg) had statistically difference (P<0.01 or P<0.05) in body weight compared with the positive reference group (V-PEG-SC-20K-rhIL-2, 125 μg/kg), the results showed that the safety of mPEG-SS-5k-GP8-high modification was better than the positive reference group (imitation NKTR214) with equal dose.

TABLE 33
Body weight growth rate of animals in each group (Female, Mean ± SEM)

| | | Mean body weight | | On the 3rd day after |
|---|---|---|---|---|
| | | On the day of | On the 3rd day | the last dose(D32) |
| | | the first | after the last | weight growth |
| Groups | n | dose(D1) | dose(D32) | rate(%) |
| Negative control group | 8 | 22.11 ± 0.26 | 23.89 ± 0.45 | 8.01 |
| Positive control group(Quanqi)- | 8 | 22.10 ± 0.23 | 22.34 ± 0.37* | 1.15 |
| 1 million IU/kg | | | | |
| V-PEG-SC-20k-rhIL-2-125 μg/kg | 8 | 22.11 ± 0.30 | 20.74 ± 0.40** | −6.20 |
| mPEG-SS-5K -GP8- high | 8 | 22.10 ± 0.29 | 21.61 ± 0.65* | −2.20 |
| modification high dose- | | | | |
| 250 μg/kg | | | | |
| mPEG-SS-5K -GP8- high | 8 | 22.11 ± 0.29 | 22.55 ± 0.25*^{ΔΔ} | 2.08 |
| modification medium dose- | | | | |
| 125 μg/kg | | | | |
| mPEG-SS-5K -GP8- high | 8 | 22.11 ± 0.27 | 22.60 ± 0.40* | 2.26 |
| modification low | | | | |
| dose-62.5 μg/kg | | | | |

Note:
**P < 0.01, vs negative control; *P < 0.05, vs negative control.
^{ΔΔ}P < 0.01, vs positive reference (imitation NKTR214).

TABLE 34
Body weight growth rate of animals in each group (Female, Mean ± SEM)

| | | | | On the 16th day |
|---|---|---|---|---|
| | | Mean body weight | | after the last |
| | | On the day of | On the 16th day | dose(D45) |
| | | the first | after the last | weight growth |
| Groups | n | dose(D1) | dose(D45) | rate(%) |
| Negative control group | 8 | 22.11 ± 0.26 | 24.36 ± 0.35 | 10.28 |
| Positive control group(Quanqi)- | 8 | 22.10 ± 0.23 | 23.24 ± 0.36* | 5.21 |
| 1 million IU/kg | | | | |
| V-PEG-SC-20k-rhIL-2-125 μg/kg | 8 | 22.11 ± 0.30 | 21.45 ± 0.87** | −3.14 |
| mPEG-SS-5K -GP8- high | 8 | 22.10 ± 0.29 | 23.78 ± 0.49 | 7.60 |
| modification high dose- | | | | |
| 250 μg/kg | | | | |
| mPEG-SS-5K -GP8- high | 8 | 22.11 ± 0.29 | 23.78 ± 0.35^{Δ} | 7.67 |
| modification medium | | | | |
| dose-125 μg/kg | | | | |
| mPEG-SS-5K -GP8- high | 8 | 22.11 ± 0.27 | 23.91 ± 0.49 | 8.19 |
| modification low dose- | | | | |
| 62.5 μg/kg | | | | |

Note:
**P < 0.01, vs negative control; *P < 0.05, vs negative control.
^{Δ}P < 0.05, vs positive reference (imitation NKTR214).

3. Analysis of results

mPEG-SS-5k-GP8-high modification significantly inhibited the tumor growth in A375 human melanoma model at high and medium doses (250 μg/kg, 125 μg/kg), and mPEG-SS-5K-GP8-high modification showed a trend of tumor inhibition at low dose (62.5 μg/kg). The inhibition of tumor growth by mPEG-SS-5k-GP8-high modification was in good dose-dependent. Positive reference group (V-PEG-SC-20k-rhIL-2, imitation NKTR214) at 125 μg/kg can significantly inhibit the growth of tumor in A375 human melanoma model, positive control group (Quanqi) showed a trend of tumor inhibition in A375 human melanoma model at 1 million IU/kg.

The animals in mPEG-SS-5K-GP8-high modification (high dose) group (250 μg/kg) and V-PEG-SC-20k-rhIL-2 group (125 μg/kg) showed basically tolerance to the treatment. The animals in mPEG-SS-5k-GP8-high modification with medium and low dose (125 μg/kg, 62.5 μg/kg) and positive control group (Quanqi, 1 million IU/kg) showed good tolerance to the treatment. No animal death was observed in each treatment group.

The results showed that with similar tumor suppression effect, animals in mPEG-SS-5k-GP8-high modification groups were more tolerable than that in V-PEG-SC-20k-rhIL-2 group. With the similar safety, the total dose of positive control group (Quanqi) was higher than that of mPEG-SS-5K-GP8-high modification (high dose) group, but the drug effect of mPEG-SS-5K-GP8-high modification was better.

### Example 17: Pharmacodynamic Evaluation of PEG-Modified IL-2 Mutant in A498 Human Renal Cancer Model

### I. Experimental Methods

A498 cells were cultured in DMEM medium containing 10% fetal bovine serum. A498 cells in exponential growth phase were collected and resuspended in PBS. hu-PBMCs (human peripheral blood mononuclear cells) were cultured in 1640 medium containing 10% fetal bovine serum. PBMCs were collected at the third day after stimulated with OKT-3 and IL-2, and resuspended in PBS. 5×10⁶ cells/0.1 ml A498 cells and 5×10⁶ cells/0.1 ml PBMCs were mixed in equal volume, and were inoculated subcutaneously into the right flank of NOD/SCID mice, 0.2 mL per mouse. On the day after cell inoculation, the animals were grouped according to body weight, and the first dose was started on the day of grouping; the detailed dosage regimen and administration route were shown in the table below.

TABLE 35
The animal grouping and tumor cell inoculation information

| | | | | The | | |
|---|---|---|---|---|---|---|
| | | | | volume of | | |
| | | | | drug | Administration | Administration |
| Groups | | N | Dose | administered | route | cycle |
| 1 | Negative control group | 8 | Normal | — | — | — |
| | | | saline | | | |
| | | | for | | | |
| | | | injection | | | |
| 2 | Positive control group(Quanqi)- | 8 | 712500 | 10 mL/kg | i.v. | Once a day, |
| | 712500 IU/kg | | IU/kg | | | for 20 days |
| 3 | V-PEG-SC-20k-rhIL-2- | 8 | 125 μg/ | | | Once a |
| | 125 μg/kg | | kg | | | week, for 5 |
| 4 | mPEG-SS-5K-GP8-high | 8 | 125 μg/ | | | weeks |
| | modification | | kg | | | |
| | medium dose-125 μg/kg | | | | | |
| 5 | mPEG-SS-5K-GP8-high | 8 | 62.5 μg/ | | | |
| | modification | | kg | | | |
| | low dose-62.5 μg/kg | | | | | |

Note:
N: number of animals;
i.v.: intravenous administration.
The day of cell inoculation was D1. In terms of IL-2 activity units, the total dose of positive control group (Quanqi) was 2 times that of mPEG-SS-5K-GP8-high modification (medium dose) group.

The detection of general clinical observations, body weight, tumor volume, and tumor weight were the same as in Example 11.

### II. Experimental Results

### 1, Efficacy:

The mean tumor volume of the negative control group was 1,960.68±398.28 mm³ at the 61st day after cell inoculation.

The mean tumor volumes of mPEG-SS-5K-GP8-high modification in moderate and low dose groups (125 μg/kg, 62.5 μg/kg) were 18.99±18.99 mm³ and 248.19±209.86 mm³ at the 61st day after cell inoculation, respectively. Compared with negative control group, there were significant differences in above groups (P<0.01).

The mean tumor volume of the positive control group (Quanqi, 712,500 IU/kg) was 290.85±145.96 mm³ at the 61st day after cell inoculation, which was significantly different from that of the negative control group (P<0.01). Results showed that mPEG-SS-5K-GP8-high modification at moderate dose, which was only ½ dose of Quanqi, had better efficacy than positive control group (Quanqi).

The mean tumor volume of V-PEG-SC-20k-rhIL-2 group (125 μg/kg) was 110.22±63.15 mm³ at the 61st day after cell inoculation, which was significantly different from that of the negative control group (P<0.01). There was no significant difference in tumor volume between mPEG-SS-5K-GP8-high modification (moderate dose) and positive reference group (P>0.05). The results showed that with the same dose, mPEG-SS-5K-GP8-high modification was more effective than the positive reference (V-PEG-SC-20k-rhIL-2, imitation NKTR214).

The results of tumor weight were basically consistent with the results of tumor volume. The TGI of each group was calculated based on tumor weight, and the TGI of mPEG-SS-5k-GP8 (moderate dose) group and (low dose) group were 998, 85% respectively. The TGI of positive control group (Quanqi) and positive reference group (V-PEG-SC-20k-rhIL-2, imitation NKTR214) were 83% and 93%, respectively.

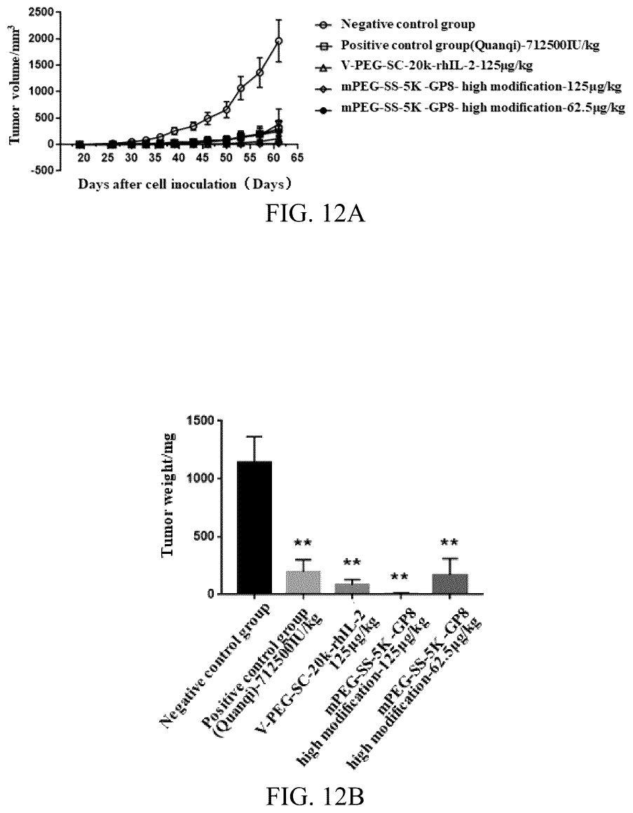

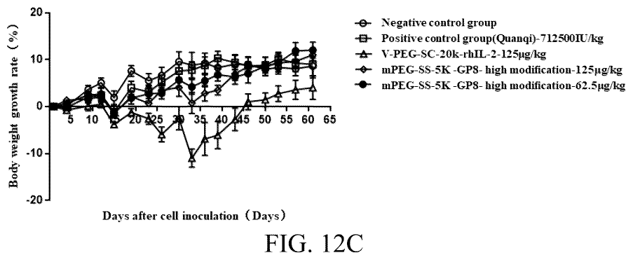

The experimental results were shown in the table below and FIG. 12:

TABLE 36
Tumor volume of animals in each group at the 61st day after inoculation
(Female, Mean ± SEM)

| | | at the 61st day after cell inoculation | |
|---|---|---|---|
| | | | P value(vs |
| Groups | n | Tumor volume(mm³) | Model) |
| Negative control group | 8 | 1,960.68 ± 398.28 | — |
| Positive control | 8 | 290.85 ± 145.96** | 0.001 |
| group(Quanqi)-712500 IU/kg | | | |
| V-PEG-SC-20k-rhIL-2- | 8 | 110.22 ± 63.15** | 0.000 |
| 125 μg/kg | | | |
| mPEG-SS-5K -GP8- | 8 | 18.99 ± 18.99** | 0.000 |
| high modification | | | |
| moderate dose -125 μg/kg | | | |
| mPEG-SS-5K -GP8- | 8 | 248.19 ± 209.86** | 0.020 |
| high modification | | | |
| low dose -62.5 μg/kg | | | |

Note:
**P < 0.01, vs negative control; *P < 0.05, vs negative control.

TABLE 37
Tumor weight of animals in each group at the 61st day after inoculation
(Female, Mean ± SEM)

| | | At the 61st day after cell inoculation | | | |
|---|---|---|---|---|---|
| | | | T/C | | P value(vs |
| Groups | n | tumor weight(mg) | (%) | TGI(%) | Model) |
| Negative control group | 8 | 1,139.71 ± 223.35 | — | — | — |
| Positive control | 8 | 196.01 ± 104.77** | 17 | 83 | 0.002 |
| group(Quanqi)-712500 IU/kg | | | | | |
| V-PEG-SC-20k-rhIL-2 group- | 8 | 82.38 ± 48.68** | 7 | 93 | 0.000 |
| 125 μg/kg | | | | | |
| mPEG-SS-5K -GP8- high | 8 | 7.19 ± 7.19** | 1 | 99 | 0.000 |
| modification moderate dose- | | | | | |
| 125 μg/kg | | | | | |
| mPEG-SS-5K -GP8- high | 8 | 167.33 ± 141.47** | 15 | 85 | 0.002 |
| modification low dose -62.5 μg/kg | | | | | |

Note:
**P < 0.01, vs negative control; *P < 0.05, vs negative control.

### 2. Safety:

In mPEG-SS-5k-GP8-moderate dose group (125 μg/kg), 1 animals showed moderate body weight loss during the treatment, and the animals in the group were generally tolerant to the treatment. mPEG-SS-5k-GP8-high modification at low dose (62.5 μg/kg) did not show obvious drug toxicity during the treatment, and the animals were well tolerated during the treatment.

The animals in the positive control group (Quanqi, 712,500 IU/kg) showed no obvious drug toxicity during the treatment and were well tolerated during the treatment.

In the V-PEG-SC-20k-rhIL-2 group (125 μg/kg), 3 animals showed moderate weight loss, and 1 animal showed moderate to severe weight loss during the treatment, and the animals in the group were basically tolerant to the treatment. Compared with V-PEG-SC-20k-rhIL-2 (125 μg/kg), mPEG-SS-5K-GP8-high modification at moderate dose group (125 μg/kg) had statistically significant difference in body weight on the 4th day after the last dose (D333) (P<0.01). The results showed that the safety of mPEG-SS-5K-GP8-high modification was better than that of positive reference group (imitation NKTR214) with the same dose.

TABLE 38
Body weight growth rate of animals in each group (Female, Mean ± SEM)

| | | Mean body weight | | |
|---|---|---|---|---|
| | | On the day of | On the 4th day | On the 4th day after |
| | | the first | after the last | the last dose(D33) |
| Groups | n | dose(D1) | dose(D33) | Weight gain rate(%) |
| Negative control group | 8 | 22.10 ± 0.29 | 24.03 ± 0.58 | 8.80 |
| Positive control group(Quanqi)- | 8 | 22.11 ± 0.28 | 23.81 ± 0.31 | 7.78 |
| 712500 IU/kg | | | | |
| V-PEG-SC-20k-rhIL-2-125 μg/kg | 8 | 22.11 ± 0.27 | 19.66 ± 0.42** | −11.03 |
| mPEG-SS-5K -GP8- high | 8 | 22.11 ± 0.25 | 22.28 ± 0.53*^{ΔΔ} | 0.75 |
| modification moderate dose- | | | | |
| 125 μg/kg | | | | |
| mPEG-SS-5K -GP8- high | 8 | 22.10 ± 0.24 | 23.01 ± 0.33 | 4.18 |
| modification low dose- | | | | |
| 62.5 μg/kg | | | | |

Note:
**P < 0.01, vs negative control; *P < 0.05, vs negative control.
^{ΔΔ}P < 0.01, vs positive reference (imitation NKTR214).

TABLE 39
Body weight growth rate of animals in each group (Female, Mean ± SEM)

| | | Mean body weight | | On the 28th day |
|---|---|---|---|---|
| | | On the day of | On the 28th day | after the last |
| | | the first | after the last | dose(D61) |
| Groups | n | dose(D1) | dose(D61) | Weight gain rate(%) |
| Negative control group | 8 | 22.10 ± 0.29 | 23.99 ± 0.43 | 8.62 |
| Positive control group(Quanqi)- | 8 | 22.11 ± 0.28 | 24.05 ± 0.43 | 8.98 |
| 712500 IU/kg | | | | |
| V-PEG-SC-20k-rhIL-2-125 μg/kg | 8 | 22.11 ± 0.27 | 23.00 ± 0.60 | 4.02 |
| mPEG-SS-5K -GP8- high | 8 | 22.11 ± 0.25 | 24.55 ± 0.45 | 11.00 |
| modification moderate dose- | | | | |
| 125 μg/kg | | | | |
| mPEG-SS-5K -GP8- high | 8 | 22.10 ± 0.24 | 24.75 ± 0.37 | 12.04 |
| modification low dose- | | | | |
| 62.5 μg/kg | | | | |

### 3. Analysis of Results

mPEG-SS-5k-GP8-high modification can significantly inhibit the tumor growth in A498 human renal cancer model at moderate and low doses (125 μg/kg, 62.5 μg/kg). The inhibition of tumor growth by mPEG-SS-5k-GP8-high modification was dose-dependent. V-PEG-SC-20k-rhIL-2 at the dose of 125 μg/kg and positive control group (Quanqi) at the dose of 712,500 IU/kg could significantly inhibit the growth of tumor volume in A498 human renal cancer model.

The animals in mPEG-SS-5K-GP8-high modification group at moderate dose (125 μg/kg) and V-PEG-SC-20k-rhIL-2 group (125 μg/kg) showed basically tolerance to the treatment. Animals in mPEG-SS-5K-GP8-high modification group at low dose (62.5 μg/kg) and positive control group (Quanqi, 712500 IU/kg) were well tolerated to the treatment. No animal death was observed.

The results indicated that with similar tumor suppression effect, animals in mPEG-SS-5k-GP8-high modification group were more tolerable than that in V-PEG-SC-20k-rhIL-2 group.

### Example 18: Activation Effect of PEG Modified IL-2 on Cynomolgus Monkey PBMCs

The cynomolgs (3 cynomolgs in each group) were intravenously injected with different doses of mPEG-SS-5K-GP8-high modification (0.01 mg/kg, 0.03 mg/kg), and the blank control group was given vehicle control, once a week, a total of 5 doses (D1, D8, D15, D22, D29). Thirty days after the first dose (D1), peripheral blood was collected, peripheral blood mononuclear cells (PBMCs) were isolated and marked with anti-CD25-APC fluorescent antibody (Miltenyi, product number 130-113-842), detected by FACS.

TABLE 40
FACS detection of the CD25 expression
in PBMCs of cynomolgus monkey

| | | Positive rate of | |
|---|---|---|---|
| Groups | Animal No. | CD25 | Mean ± SD |
| blank | 6 | 6.7% | 8.1% ± 2.6% |
| control | 7 | 11.1% | |
| | 8 | 6.5% | |
| 0.01 mg/kg | 16 | 14.2% | 9.6% ± 4.0% |
| | 17 | 7.3% | |
| | 18 | 7.4% | |
| 0.03 mg/kg | 26 | 15.3% | 16.3% ± 2.3%* |
| | 27 | 18.9% | |
| | 28 | 14.6% | |

Note:
*P < 0.05 compared with the blank control group, there was significant difference.

The above data showed that the expression of CD25 in 0.03 mg/kg group was significantly different from that in the blank control group. CD25 was a marker of T cell activation in PBMCs, which proved that T cells were effectively activated by mPEG-SS-5K-GP8-high modification at the dose of 0.03 mg/kg.

Example 19 Dose Exploration Experiment of Repeated Intravenous Injections in Cynomolgus Monkeys for 4 Weeks

Cynomolgus monkeys were intravenously injected with different doses of mPEG-SS-5K-GP8-high modification for several times, and the possible toxic reactions during treatment were observed. There were four groups at the dose of 0.03, 0.1, 0.3, 0.5 mg/kg in this experiment. The 0.03 mg/kg group was increased to 0.3 mg/kg after 3 doses of 0.03 mg/kg and served as 0.3 mg/kg group, for a total of six doses.

### I. Grouping and Experimental Design

According to the table below, six animals were randomly divided into four groups according to sex and body weight

TABLE 41
Experiment design and grouping

| | | | The volume | Concentration | | |
|---|---|---|---|---|---|---|
| | | | of drug | | | |
| | | Dose | administered | of drug | Animals | |
| Groups | | mg/kg | ml/kg | administered mg/ml | Number | Gender |
| 1 | Low dose | 0.03 | 0.5 | 0.06 | 2 | male: 1 |
| | | | | | | female: 1 |
| 2 | Moderate dose | 0.1^{a} | 0.5 | 0.2 | 2 | male: 1 |
| | | | | | | female: 1 |
| 3 | Second high dose | 0.3^{b} | 0.5 | 0.6 | 2 | male: 1 |
| | | | | | | female: 1 |
| 4 | High dose | 0.5^{c} | 0.5 | 1 | 2 | male: 1 |
| | | | | | | female: 1 |

^{a}The first dose of group 1 and 3 was performed 21 days after the first dose of group 2;
^{b}Animals of group 1 was given 0.3 mg/kg during D29-D43;
^{c}The second dose was not performed, and a total of 4 doses were completed.

The clinical observations of the animals were conducted daily during the experiment. The changes of body weight, food intake, body temperature and blood pressure were monitored, and the clinical pathology, immunocyte phenotype analysis, cytokines and general observation were detected.

### II. Results

There was no death in cynomolgus monkeys in the groups of 0.03, 0.1, 0.3 and 0.5 mg/kg.

At the dose of 0.03 mg/kg, cynomolgus monkeys did not show abnormalities and showed a trend of weight gain.

At the dose of 0.1 mg/kg, the spleen of males became larger, the ratio of CD56+ cells decreased, and the body weight showed an increasing trend.

At the dose of 0.3 mg/kg, the feed intake decreased, but the body weight increased. The gross anatomy showed that the spleen of animals were enlarged. Clinical pathological examination showed increased WBC count, decreased red blood cell count, decreased hematocrit, decreased platelet count and total protein.

At the dose of 0.5 mg/kg, the animals had adverse reactions such as lethargy, decreased spontaneous activity, excessive loose stools, and visualization mucosa pale after the first dose. The administration was suspended once, and the administration was completed for 4 doses total, and the feed intake and body weight of the animals decreased. Gross anatomy showed symptoms such as enlarged spleen, smaller thymus, abdominal distension, and generalized skin relaxation. Clinical pathological examination showed increase of WBC count, neutrophils (absolute value), monocytes (absolute value), eosinophil (absolute value), basophils (absolute value), urea, creatine kinase, fibrinogen, and showed decrease of RBC count, hematocrit, platelet count, total protein and the ratio of CD56+ cells.

Preliminary results showed that cynomolgus monkeys at the dose of 0.5 mg/kg showed severe adverse reactions without death.

In addition, according to the literature related to NKTR-214 (NKTR-214, an Engineered Cytokine with Biased IL2 Receptor Binding, Increased Tumor Exposure, and Marked Efficacy in Mouse Tumor Models): in cynomolgus monkeys administered every 14 days for 4 doses, the maximum tolerated dose (MTD) was 0.1 mg/kg, which resulted in 24-fold increase in CD25+ and 4-fold increase in total lymphocytes. Compared with NKTR-214, the mPEG-SS-5k-GP8-high modification showed no serious adverse reaction at shorter dosing interval (7 days), more administration times (5 times), higher drug dose (0.3 mg/kg), suggesting that mPEG-SS-5K-GP8-high modification had predominance in safety and tolerability.

1-18. (canceled)

19. A human interleukin-2 mutant, which does not comprise an adjacent lysine.

20. The human interleukin-2 mutant of claim 19, wherein one, two or more amino acids at position 1, 8, 9, 18, 19, 48, 49, 72 or/and 81 from the N-terminus are substituted compared with wild-type human IL-2; the amino acid sequence of the wild-type human IL-2 is as shown in SEQ ID NO: 1.

21. The human interleukin-2 mutant of claim 19, wherein the amino acid at position 8 or/and 9 from the N-terminus is substituted compared with wild-type human IL-2; or the amino acid at position 48 or/and 49 from the N-terminus is substituted compared with wild-type human IL-2; the amino acid sequence of the wild-type human IL-2 is as shown in SEQ ID NO: 1.

22. The human interleukin-2 mutant of claim 19, wherein each mutation is respectively selected from the following residue substitutions:
position 1: A1 deletion; position 8: K8R; position 9: K9R; position 18: L18M; position 19: L19S; position 48: K48W, R; position 49: K49R; position 72: L72F; position 81: R81D.

23. The human interleukin-2 mutant of claim 19, wherein the mutation is selected from the mutation schemes in the table below:

| Mutant name | mutation site |
|---|---|
| IL-2 | — |
| G438P1 | K8R/L18M/L19S/K48W |
| G438P8 | K8R/K48W/L72F/R81D |
| G438P12 | K8R/L18M/L19S/K48R |
| G438P20 | K9R/L18M/L19S/K48R |
| G438P21 | Deletion of the amino acid A in position 1, |
| | K9R/L18M/L19S/K48R |
| G438P22 | K8R/L18M/L19S/K49R |

24. A polyethylene glycol modified product of human interleukin-2, which uses polyethylene glycol succinimidyl succinate as PEG modifier, and an average of 4.5 to 8.5 PEG molecules are conjugated to each one human interleukin-2, wherein the human interleukin-2 is wild-type human interleukin-2 or the human interleukin-2 mutant of claim 19.

25. The polyethylene glycol modified product of claim 24, wherein the PEG modifier is straight-chain polyethylene glycol succinimidyl succinate.

26. The polyethylene glycol modified product of claim 24, wherein the molecular weight of the PEG modifier is 5-20 kDa.

27. The polyethylene glycol modified product of claim 24, when the molecular weight of the PEG modifier is 5 kDa, an average of 5.5 to 7.5 PEG molecules are conjugated to each one human interleukin-2; when the molecular weight of the PEG modifier is 10-20 kDa, an average of 6.5 to 8.5 PEG molecules are conjugated to each one human interleukin-2.

28. The polyethylene glycol modified product of claim 24, wherein the structure of the PEG modifier is shown as follows: 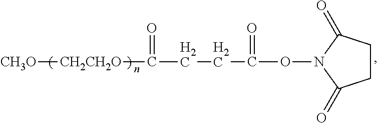
wherein n is an integer from 97 to 494.

29. The polyethylene glycol modified product of claim 24, wherein the structure of the polyethylene glycol modified product of human interleukin-2 is as follows: 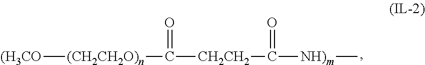
wherein n is an integer from 97 to 494, and m is 4.5 to 8.5.

30. A method for treating a neoplastic disease using the polyethylene glycol modified product of human interleukin-2 of claim 24.

31. The method of claim 30, wherein the neoplastic disease comprises squamous cell carcinoma, melanoma, colon cancer, breast cancer, ovarian cancer, prostate cancer, stomach cancer, liver cancer, small cell lung cancer, non-small cell lung cancer, thyroid cancer, kidney cancer, bile duct cancer, brain cancer, cervical cancer, maxillary sinus cancer, bladder cancer, esophageal cancer, Hodgkin's disease, and adrenocortical cancer.

32. A composition for treating neoplastic disease, which comprises the polyethylene glycol modified product of human interleukin-2 of claim 24, and further comprises a HER2 antibody, a PD-1 antibody, a PD-L1 antibody, or a CD26 antibody.

33. A composition for treating neoplastic disease, which comprises the human interleukin-2 mutant of claim 19, and further comprises a HER2 antibody, a PD-1 antibody, a PD-L1 antibody, or a CD26 antibody.

34. A method for realizing the controlled release and sustained release of the activity of bioactive molecules, wherein the method is to modify the amino group of the bioactive molecule with polyethylene glycol modifier, and the activity of the bioactive molecule is gradually released as the PEG gradually falls off from the modified product, wherein the polyethylene glycol modifier is polyethylene glycol succinimidyl succinate; wherein the bioactive molecule is interleukin-2.

35. The method of claim 34, wherein the PEG modifier is preferably straight-chain polyethylene glycol succinimidyl succinate.
